(19) United States
(12) Patent Application Publication
(10) Pub. No.: US 2008/0201172 A1
(43) Pub. Date: Aug. 21, 2008

(54) METHOD, SYSTEM AND COMPUTER SOFTWARE FOR USING AN XBRL MEDICAL RECORD FOR DIAGNOSIS, TREATMENT, AND INSURANCE COVERAGE

(76) Inventor: Richard T. McNamar, Falls Church, VA (US)

Correspondence Address: FOLEY AND LARDNER LLP;SUITE 500, 3000 K STREET NW, WASHINGTON, DC, 20007, US

(21) Appl. No.: 11/740,122

(22) Filed: Apr. 25, 2007

Related U.S. Application Data
(60) Provisional application No. 60/794,533, filed on Apr. 25, 2006.
(60) Provisional application No. 60/794,821, filed on Apr. 26, 2006.
(60) Provisional application No. 60/794,835, filed on Apr. 26, 2006.
(60) Provisional application No. 60/794,838, filed on Apr. 26, 2006.
(60) Provisional application No. 60/794,836, filed on Apr. 26, 2006.
(60) Provisional application No. 60/794,858, filed on Apr. 26, 2006.
(60) Provisional application No. 60/794,822, filed on Apr. 26, 2006.
(60) Provisional application No. 60/794,834, filed on Apr. 26, 2006.
(60) Provisional application No. 60/841,529, filed on Sep. 1, 2006.
(60) Provisional application No. 60/844,674, filed on Sep. 15, 2006.
(60) Provisional application No. 60/845,777, filed on Sep. 20, 2006.
(60) Provisional application No. 60/908,050, filed on Mar. 26, 2007.

(51) Int. Cl. G06F 19/00; A61B 5/00

(52) U.S. Cl. 705/3; 600/300

(57) ABSTRACT

A healthcare management method, system and program product comprising: extracting and/or receiving or generating a medical record comprising at least clinical examination data or laboratory results and context data therefore for a patient; creating or updating an XBRL medical record comprising XBRL data fields containing clinical examination data and/or laboratory results made over a period of time and associated metadata representing attributes at the data value level based on a medical taxonomy, the creating or updating comprising converting the clinical examination data and/or laboratory results into values in one or more of the XBRL data fields and creating from the context metadata and associating the metadata representing attributes at the data value level based on the medical taxonomy and forming links between and/or among at least two items selected from one or more of the categories of the data fields, metadata and components associated with the data fields in the XBRL medical record; obtaining data made over a period of time for that patient in a given data field; performing an algorithm on the obtained data for the at least one of the data fields to obtain an algorithm calculation result; and communicating the algorithm calculation result. In a further embodiment, a genotype for a patient is obtained and used to select a data field from an XBRL medical record for the patient, and then an algorithm is performed on data made over a period of time for at least one of the data fields to obtain an algorithm calculation result, and the algorithm calculation result is communicated.

### CROSS-REFERENCE TO RELATED APPLICATIONS

This application claims priority from Provisional U.S. Application 60/794,533, filed Apr. 25, 2006, Provisional U.S. Application 60/794,858, filed Apr. 26, 2006, Provisional U.S. Application 60/794,821, filed Apr. 26, 200, Provisional U.S. Application 60/794,838, filed Apr. 26, 2006, Provisional U.S. Application 60/794,834, filed Apr. 26, 2006, Provisional U.S. Application 60/794,835, filed Apr. 26, 2006, Provisional U.S. Application 60/794,836, filed Apr. 26, 2006, Provisional U.S. Application 60/794,822, filed Apr. 26, 2006, Provisional U.S. Application 60/841,529, filed Sep. 1, 2006, Provisional U.S. Application 60/844,674, filed Sep. 15, 2006, Provisional U.S. Application 60/845,777, filed Sep. 20, 2006, from Provisional U.S. Application 60/908,050, filed Mar. 26, 2007. The entire content of the aforementioned applications are incorporated herein by reference.

### TECHNICAL FIELD

Method, systems computer software utilizing an XBRL enabled EHR (XBRL-EHR) to verbally or electronically establish, manage and periodically update a patient's electronic health record utilizing metadata by data field for information from clinical, laboratory, genomic or Picture Archiving and Communication software systems (PACS) for (1) analyzing mathematically all the patients longitudinal data to assist a physician in diagnosing and treating the patient, (2) displaying the data in the context of data from other databases or APIs (mash-ups), (3) permitting the XBRL-EHR to be transferred or viewed (in whole or part) electronically for primary or secondary uses, and (4) enabling the PHR to be used to pay insurance claims of the patient.

### DEFINITIONS

PHR (personal health record) and EMR (electronic medical record) are terms that are often used interchangeably in the healthcare literature. In this patent application, the term EMR refers to an XBRL-EHR that is an electronic health record that uses XBRL with metadata by data field within the EMR and will include PACS data.

Picture Archiving and Communication Systems (PACS) include; Ultra Sound; MRI; CR; nuclear medicine; digital fluoroscopy; digital radiology; angiography, and CT.

Genomic data refers to the individual patient's genetic or DNA data. It is defined as part of laboratory tests just as serum cholesterol levels are defined as part of laboratory tests.

Primary use of health data is to provide real-time, direct care of an individual. Secondary use of health data is non-direct care use for public health, research, or commercial purposes.

### BACKGROUND

Various surveys suggest that the small practices (one to five physicians) that account for approximately two-thirds of all physician visits and prescriptions written each year have an extremely low adoption rate for information technology, typically surveyed at 5 to 15% of the physicians. And their use is generally for billing and scheduling, not the practice of medicine itself. While larger groups, e.g., Kaiser Permanente or the Department of Veterans Affairs have a much higher use of IT, their use of IT for a PHR is relatively low and confined to their computer system in terms of transferability.

Historically, the health care providers have made good decisions in terms of their accounting, computer usage, and managing of IT capabilities. In addition, they have historically purchased computers and computer software for their patient management and billing practices that were proprietary in nature. That is, computer software program for one vendor, which computers do not operate on or with another manufacturer's computer operating system or applications software.

Today there is no interoperability of computer systems or software to transfer electronically a PHR from one health care provider (physician, clinic, hospital) to another health care provider. Well over 90 percent of the health records in America are paper based. When a patient is referred from a primary care physician to a specialist the patient's paper records are transferred by FedEx or comparable messenger service. This is true whether the transfer is across town or across the country from New York to California.

Today, typically at the check-in for a health care provider, there is seldom, if ever, an electronic record of a patient's previous health care records, past lab test results, prescriptions, etc. available to the attending physician. This is true whether it is a physician the patient has seen for many years or a new physician in the same clinic or office. Typically the records are paper files. Studies suggest that a patient's previous paper record cannot be located in a doctor's office close to 40% of the time.

Patients move geographically in America and their paper records often are not part of the current physician's diagnosis in the new location. Even when the patient does not move, the past clinical or laboratory findings are on multiple pieces of paper in one manila folder. The doctor may or may not happen to see the previous laboratory readings on multiple pages and relate them to a trend.

Even in those health care institutions that have electronic patient health records, their physicians usually can not share patient medical information electronically with other physicians, clinics, or hospitals not on the same computer software system, i.e., the PHR is not interoperable between health care providers in different organizations. This is in part because the vendors specifically did not design them to be exchanged.

The Extensible Markup Language (XML) is the universal language of the Internet to display documents. It utilizes metadata about documents to identify them. An example of metadata on documents is the pull down window that the user sees when they open a Word document and it states that the author is John Doe and the title is Sales Report and the date and time it was last used at January 1, 2007 at 10:30 AM. This is metadata about the document, but not its contents or fields of data. Typically, a PHR in XML is in the HTML format and it is simply a picture or electronic representation of the patient's medical record on a paper page.

Medical records that are in XML format are simply electronic pictures or representations of a typed paper page. They can be viewed, but not updated or analyzed automatically with the data in the picture of the document. And, they do not provide assurance of the definitions or correctness of the data within the document; therefore they are inevitably non-exchangeable or useable between two physicians. The patient's health care record was just that, a record or document. Whether the patient's information was captured in a Word file, an Excel file, a PDF, or other types of files on a computer, it was essentially transmitting a picture of a page, which is a HyperTextMarkupLanguage (“HTML”) file. The picture of the page may have the patient's data in it, but it was not accessible to computer analysis. That is, the doctor did not have a tool to look at the data on the patient's health record and relate it to any other database except the information in the physician's head, i.e., a human has to read the database, a computer can not.

As the United States population approaches 300 million, each of these individuals is a potential medical patient with a unique individual health record. Patient visits to physician are bi-modally distributed with the greatest frequency of visits for those who are very young with childhood diseases and the very old with their infirmities.

People move, change physicians, consult with second physician, change jobs, join the military, leave the military, move geographically or simply move to neighborhoods in the same city. Any of these events may cause an individual to change physicians within a geographic area or in a new geographic area. At present there is no widespread generally used system for electronically transferring an individual's patient record to a new physician. In addition to changing their physician, people are referred by physician to a specialist physician, who may be in a different clinic or practice in the same city or in an entirely different location, which necessitates transferring the patient's medical records.

Physicians retire or die, which necessitates transferring a patient's health records to the patient's new physician. Changes, moves, and referrals all generate the need to transfer a patient's previous and existing medical records to a new physician for their use and review. Even for a medical consultation by another physician, the patient's records must be sent for viewing and review by the consulting physician. Taken together, the causes suggest that the need to transfer and move a patient's medical records may happen close to one billion times per year in a country with 300 million people.

Unfortunately, health records for patients are often maintained manually in the physician's office. One estimate is that only twenty-five percent of all patients' records are computerized at this time while the rest are created, filed, and retrieved manually. Obviously, a patient's medical record that is available only in their physician's office is not accessible to an attending physician in another location, e. g., a hospital, or in an office in another state. Yet, knowledge of a patient's medical condition, current medications, allergies, etc. may be vitally important in treating the patient.

This non-standardization of health care IT was documented in the 2005 Congressional presentation, www.endingthedocumentgame.org. It focused on the number of deaths caused by the unavailability of health care information or incorrect information.

This report highlights the fact that over 100,000 medical deaths occur in the United States because the attending physicians did not have access to the current medical record of the patient they were treating. Many observers believe that the 100,000 deaths from medical information errors is greatly understated and underreported by the medical profession.

However, today patient's medical records are transferred by fax or couriered between offices. This system is expensive to the patient, cumbersome, error prone, and subject to abuse. There is clearly a need to make patient's medical records electronically transportable between physicians' offices, clinics, and hospitals.

Typical of the state-of-the-art health care software on the market today is CareEvolution, Inc. See www.careevolution.com. This Company's web site says, “CareEvolution is a leading provider of secure interoperability solutions. The CareEvolution HIEBus employs a robust Service Oriented Architecture (SOA) to enable heterogeneous EMRs to “share clinical information in a secure, reliable, and incremental manner. Distinct components such as Identity Management, Record Location, Clinical Data Integration, Audit & Log, Data Persistence, Visualization, Terminology, and Data Mining may be adopted piecemeal or as a comprehensive technology platform.” But it lacks the electronic interoperability among health care providers, and can only provide data within its own platform. This is similar to the system used at Kaiser Permanente., which is also not interoperable among software and systems of other providers, only their own “technology platform” of computer system/network. Some of the more advanced medical software applications today do use XML. On Mar. 16, 2007 Practice Fusion and Google announced a new web based free service for physicians. See: www.practicefusions.com. But again there is no interoperability.

Additionally, there are many health care payors ranging from Medicare to major insurance companies. The civilian and service military, Veterans Administration, and state and local government may also be payors of patient health care. For the insurance schemes the payments may be for a preferred provider group, group health care, straight service reimbursement or other arrangements. In the United States alone there are probably thousands of health care payors groups, etc. While the health care payors are much more sophisticated than the providers in terms of using modern technology to manage their businesses, there is a large variety of differing incompatible computer systems that are used by the payors. These systems have grown up historically and many are on antiquated proprietary software. Many are on state of the art web based XML software applications. The computer software and languages were simply not designed to be interoperable with each other. As a result, the patient's computerized health care records, when available, are typically not interoperable with the health care payors' computer systems and software.

### SUMMARY

In one embodiment, a healthcare management method, system and program product, is provided, the method comprising: extracting and/or receiving or generating a medical record comprising at least clinical examination data or laboratory results and context data therefore for a patient; creating or updating an XBRL medical record comprising XBRL data fields containing clinical examination data and/or laboratory results made over a period of time and associated metadata representing attributes at the data value level based on a medical taxonomy, the creating or updating comprising converting the clinical examination data and/or laboratory results into values in one or more of the XBRL data fields and creating from the context metadata and associating the metadata representing attributes at the data value level based on the medical taxonomy and forming links between and/or among at least two items selected from one or more of the categories of the data fields, metadata and components associated with the data fields in the XBRL medical record; obtaining data made over a period of time for that patient in a given data field; performing an algorithm on the obtained data for the at least one of the data fields to obtain an algorithm calculation result; and communicating the algorithm calculation result.

In a further embodiment, the algorithm is a regression algorithm.

In a further embodiment, the step of component is provided to automatically identifying one or more potential diagnosis based on the algorithm calculation results; and communicate the potential diagnosis.

In a further embodiment, the step or component is provided to identify one or more potential courses of treatment based on the algorithm calculation results; and communicate the potential course of treatment.

In a further embodiment, the step or component is provided of determining a patient search profile for the patient based on one or more of body mass index, patient habits, race, genotype; automatically accessing one or more medical databases comprising treatment and/or medical outcomes and identifying one or more treatments and/or medical outcomes for the patient search profile; and communicating the one or more treatments and/or medical outcomes.

In a further embodiment, the communicating the one or more treatments and/or medical outcomes comprises displaying the one or more treatments and medical outcomes correlated with those treatments.

In a further embodiment, the step or component is provided of determining a patient search profile for the patient based on one or more of body mass index, patient habits, race, genotype; automatically accessing one or more medical databases comprising one or more medical outcomes based on the algorithm calculation results and the patient search profile; and communicating the one or more medical outcomes.

In a further embodiment, the step or component is provided of accessing one or more medical databases comprising treatment and/or medical outcomes and selecting one or more treatments and/or medical outcomes based on the algorithm calculation results; and communicating the one or more of the treatments and/or medical outcomes.

In a further embodiment, the step or component is provided of automatically determining a potential diagnosis based on the algorithm calculation results; determining an insurance carrier associated with the patient and insurance coverage for one or more treatments for the potential diagnosis; and communicating the potential diagnosis and the insurance coverage for the one or more potential treatments.

In a further embodiment, the step or component is provided of at a trigger time and/or date automatically accessing the XBRL medical record for the patient; for at least one treatment and/or drug indicated in the accessed XBRL medical record as having been prescribed, extracting drug or treatment data from one or more medical databases; and communicating the drug or treatment data. In one embodiment, the extracting step obtains the treatment and/or drug data posted to the medical database after a predetermined date. In another embodiment, the predetermined date is a date of a previous visit data extraction step performed for that drug for that patient or a date of a last change to the XBRL record.

In a further embodiment, the step or component is provided of automatically monitoring one or more medical databases new data relating to at least one treatment and/or drug indicated in the XBRL medical record of the patient as having been prescribed; and communicating the new data relating to the at least one treatment and/or drug data.

In a further embodiment, the step or component is provided of electronically communicating the data for medical test data and/or medical examination results newly added or to be added to the XBRL medical record to a medical source for the medical test data and/or medical examination results or its designee; and receiving validation data from the medical source or its designee that the data reflecting the medical test data and/or medical examination results is accurate.

In a further embodiment, the step or component is provided of performing the steps on new clinical examination data or laboratory results received from a plurality of different computer systems.

In a further embodiment, the XBRL medical record for the patient includes medical treatment records for a given episode, and further comprising: matching the medical treatment records of the patient for the given episode to insurance coverage to obtain match results; and electronically communicating the match results. In one embodiment, medical treatment records for the given episode comprise at least one selected from the group of cost of room, disease treatment, tests, and doctor fee, and further comprising performing the matching step at patient checkout from a treatment location. In one embodiment, the matching step is performed on a periodic basis, and wherein the communicating the match results step comprises communicating the results to an insurance carrier providing the insurance coverage.

In a further embodiment, a method, system and program product is provided for creating a repository of medical treatments and/or medical outcomes, the method comprising: stripping or having stripped patient-identifying characteristics from patient medical data in the XBRL medical records to obtain stripped XBRL medical data; aggregating the stripped XBRL medical data with other stripped XBRL medical data into medical treatment and/or medical outcomes repository; and granting electronic access to the medical treatment and/or medical outcomes repository.

In a further embodiment, a method, system and program product for determining fulfilling medical drug refills is provided, the method comprising accessing XBRL patient medical records comprising data fields containing clinical examination data and/or laboratory results made over a period of time and associated metadata representing attributes at the data value level based on a medical taxonomy; selecting a given patient and determining that the patient has been prescribed or otherwise given a supply of medicine with a prescribed dosage; calculating or otherwise determining a first date when the supply of medicine will reach a predetermined level; and automatically on a second date generating a communication to at least one of the patient, a prescribing doctor, and a medicine supplier relating to the supply of medicine.

In one embodiment, the second date is a date a prescribed number of days before the first date. In a further embodiment, the communication is by an e-mail or text message inquiring whether the patient has completed the treatment and requesting a response or reply e-mail. In a further embodiment the communication is a reminder to renew a prescription or obtain a refill of the medicine.

In a further embodiment a healthcare management method, system and program product is provided based on genotype, the method comprising: obtaining a genotype for a patient; selecting based on the genotype a data field from an XBRL medical record for the patient maintained in a patient medical repository of patient medical records comprising data fields containing clinical examination data and/or laboratory results made over a period of time and associated metadata representing attributes at the data value level based on a medical taxonomy and forming links between and/or among at least two items selected from one or more of the categories of the data fields, metadata and components associated with the data fields in the XBRL medical record; performing an algorithm on data made over a period of time for at least one of the data fields to obtain an algorithm calculation result; and communicating the algorithm calculation result.

In one embodiment, the algorithm is a regression algorithm. In another embodiment, the algorithm is a comparison algorithm.

In one embodiment, the steps and/or components are provided of determining from the algorithm calculation results whether indicators are present that the patient is suffering or may in the future suffer from a particular disease; and communicating a disease result from this determining step.

In one embodiment, the steps and/or components are provided of determining one or more potential courses of treatment based on the disease result; and communicating the one or more potential courses of treatment.

In one embodiment, the steps and/or components are provided of determining an insurance carrier associated with the patient and insurance coverage for one or more treatments for the disease result; and communicating the insurance coverage for the one or more potential courses of treatments.

In one embodiment, the steps and/or components are provided of determining a medicine dosage based at least in part on the genotype; and communicating the medicine dosage.

In one embodiment, the communicating the medicine dosage comprises displaying the medicine dosage.

In a further embodiment, a method, system and program product is provided for accurately prescribing medicine, the method comprising receiving or inputting prescription input data of a new medicine prescription or a refill; accessing XBRL patient medical record comprising data fields containing medicines prescribed over a period of years for the patient and associated diagnoses and associated metadata representing attributes at the data value level based on a medical taxonomy; processing or having processed the prescription input data, wherein the processing comprises one from the group of updating the XBRL patient medicine record with the prescription input data, comparing the prescription input data to a measure of comparison, and a side effects database for prescription drugs, and communicating the prescription input data.

In one embodiment, the medical taxonomy includes one or more predetermined dosages or a range of dosages for a given medicine referenced in the prescription input data, and wherein the dosage in the prescription input data for the given medicine is not entered if it is not one of the one or more predetermined dosages or within the prescribed range without an exception.

### BRIEF DESCRIPTION OF THE DRAWINGS

FIG. 1 is a schematic block drawing showing transfer of medical records to create an XBRL medical record.

FIG. 2 is a schematic block drawing showing transfer of medical records to update an XBRL medical record.

FIG. 3 is a schematic block drawing showing transfer of medical records to form an XBRL medical record and a further transfer to a receiving organization.

FIG. 4 is a schematic block drawing showing an algorithm calculation based on longitudinal data.

FIG. 5 is a schematic block drawing showing use of algorithm calculation results and a comparison to a reference group, followed by display and/or storage and/or transmission.

FIG. 6 is a schematic block drawing showing de-identifying XBRL data and secondary transmission.

FIG. 7 is a schematic block drawing showing a claims payment process using XBRL medical records.

FIG. 8 is a schematic block drawing showing transfer of medical records.

FIG. 9 is a schematic block drawing showing another embodiment of a transfer of medical records.

FIG. 10 is a schematic block drawing showing another embodiment of a transfer of medical records.

FIG. 11 is a schematic block drawing showing a scrubbing of medical records and their transfer.

FIG. 12 is a schematic block drawing showing another embodiment of a transfer of medical records.

FIG. 13 is a schematic block drawing showing a prior art process for claims management.

FIG. 14 is a schematic block drawing showing an embodiment of a transfer of medical records for claims management/payment purposes.

FIG. 15 is a schematic block drawing showing another embodiment of a transfer of medical records for claims management/payment purposes.

FIGS. 16A and 16B comprise a schematic block diagram showing an operation to match treatments to a patient's genotype.

FIG. 17 is a schematic block diagram of an embodiment of the invention.

FIG. 18 is a schematic block diagram of an embodiment of the invention based on genotype.

### DETAILED DESCRIPTION OF THE EMBODIMENTS

The present system, method and computer software uses XBRL metadata on the individual data fields of an XBRL-HER to access medical data generated by a series of different healthcare providers over a substantial period of time, from a plurality of different incompatible systems and operating systems as well as repositories that may include distributed databases, and perform one or more algorithms on the medical data to obtain diagnosis, treatments and facilitate insurance payment. The system permits physicians or health care professionals to verbally or electronically establish, manage, and periodically update a patient's XBRL-EHR utilizing data from clinical, genomic, laboratory, or electronic PACS testing/imaging for: analyzing mathematically all the patients longitudinal data; transmitting, storing, and displaying the data alone or in the context of data from other databases or APIs (mash-ups); permitting the XBRL-EHR to be transferred (in whole or in part) electronically to any other accredited health care organization; and, permitting the XBRL-EHR to be used to pay insurance claims of the patient.

It should be noted that the term “XBRL” for purposes of this application, is given a special definition that encompasses not only XBRL, but also extensible Mark-up language equivalents that associate components representing attributes at the data value level based on a medical taxonomy and forming links between and/or among at least two items selected from one or more of the categories of the data fields, metadata and components associated with the data fields in the XBRL medical record, e.g., between two data field items, or between two metadata items, or between two component items, or between a data field item and an item of metadata, or between a data field item and a component item. By way of example, attributes at the data value level might relate to what the value represents, e.g., a systolic blood pressure value, a measurement scale, a point in time when the measurement was taken, e.g., 4 pm in the afternoon, a date when the measurement was taken, e.g., Feb. 2, 2006, information about medicines and/or treatments the patient was undergoing at the time of the measurement.

XBRL provides metadata about each specific data field and content contained within a document by utilizing XBRL components. Thus XBRL enables components to be applied by data field the most granular level. as opposed to the document level with XML. XBRL operates at the granular level.

XBRL can be implemented to be a standards-based vendor-independent technology. XBRL medical file are defined by Metadata set out in Taxonomies. Taxonomies capture the definition of individual reporting elements as well as the relationships between elements within taxonomy and in other taxonomies. Taxonomies are a collection of XML schema documents. Subsequent extensions or adjustments to the taxonomies would all be standardized. An XBRL component includes the resources necessary to implement a taxonomy for a data field, such as metadata, elements, tuples, linkbases, and stylesheets, to name a few. Schema defines items (data) and Tuples (concepts). Linkbases are a collection of links that arc concepts to resources. Style Sheets relate to rendering of data/text. The Instance Document holds business facts, contexts, units, and references. For example, an IRS 1040 Form can be represented with an XBRL taxonomy. An individual's completed 1040 is like an XBRL Instance Document. Selected links may be formed between and among the data fields and between and among metadata for the different data fields, and between and among each component (elements, tuples, metadata, and other resources) of the XBRL information taxonomy either at the time of conversion into XBRL or at a later time by means of a linking program.

A taxonomy for medical data could be implemented, for example, by multiple RHIOs, clinics, hospitals, and government agencies designing a taxonomy for the values or ranges of values that would be measured for a given data field. The taxonomy developers would typically also build in a plurality of conformance tests on each data item to insure that each data item entered/matched into a data field was within its appropriate data field parameter location. These conformance test would comprise the application of one or more business rules to specify what data could go into a data field and not accepting anything that is not supposed to be there. For example, if the data field is supposed to contain blood pressure data that is three digits and is not a decimal or a negative number, then the XBRL software will test and validate the incoming data to be sure the key punch operator or another data stream isn't entering a currency value in “dollars.

Further, XBRL allows a robust method of expressing semantic meanings for its data fields, and the relationships between those data fields. This semantic meaning can be expressed by calculations to handle summations, formulas utilizing different data fields, and may also be obtained by a definition linkbase. Thus, another conformance test could include whether the data fields A, B and C, when utilized in a calculation such as A=B+C, give the appropriate results. Thus, data is not just tested to ensure it meets specific formats, but also to ensure that the context of the data, both as a single data item and in relation to other data items, is correct as well.

Because it is the only data standard designed to work with all operating and application software, XBRL can tie together the existing legacy systems in any and all health care providers. No existing software need be replaced with new software just to obtain interoperability. All legacy software is mapped to work together using XBRL as a link or connector.

In embodiments of the invention, an XBRL file is used to access data and metadata within an XBRL file and subject it to operations such as addition, subtraction, division, multiplication, or comparison with other data by a computer. This means that the data within a patient's electronic health record is now understandable to the computer and can be managed on the computer.

Using the present system, in one embodiment data within multi-year clinical and laboratory records can be accessed and analyzed. This is in contrast to a patient's medical record in XML, which is essentially a picture of the printed page designed to be viewed by humans, but the data in it is inactive. It must be extracted, transferred, and entered into a new computer program. In the present system, XBRL-EHR software can monitor changes in clinical or laboratory values within the XBRL medical record from year to year within the medical record's data field without removing it from the patient's file. It is “Interactive.”

For example, the system can monitor a progression of Prostate-specific antigen (PSA) test scores for a male patient so that a year to year increase from 0.8 to 1.2 can now be detected using an appropriate algorithm. For example, the metadata representing laboratory results on the PSA test over a period of time can be accessed and a regression algorithm performed and the physician alerted to the results. With the present system, this type of monitoring software is not only possible, but also the doctor and the patient can be sent an e-mail informing each of the updated test results.

Monitoring can be done of other clinical and laboratory values and the findings displayed within expected ranges for outcomes. To illustrate, XBRL medical records and an automatic regression analysis permit a doctor to show a patient a printout of a computer prepared graph of the patient's cholesterol levels over their adult lifetime, regardless of geographic location of the doctor or laboratory that did the original analysis or the locations of the subsequent updates to the testing. All the data would be available in the XBRL-EMR and the regression analysis software will calculate the percentage rate of increase over any period of time. These trends can be electronically displayed by age cohort, selected population group or predicted medical outcome by probability to assist the doctor and patient in selecting a course of treatment.

Similar examples for performing automatic analysis for multi-period or longitudinal data exist for values of cholesterol levels, hormone changes, blood pressure, sugar levels and the like. Paper records simply do not permit this type of automatic pre-determined regression analysis of multi-period data to be undertaken.

Note that a treatment is something one does to determine or cause an outcome. For example, if one elects a [treatment] not to stop smoking, the outcome is that that person has an X probability of dying of lung cancer in Y years or his/her life expectancy if he/she keeps smoking is Z. In one embodiment, a regression analysis of observations to date on data from a particular data field is extrapolated over time and the extrapolated results displayed against a background of data that are the medical outcomes. By way of example, assume that two variants are blood pressure and weight or body mass in the regression analysis. If one has the data to calculate the regression analysis on patient A's blood pressure from age 40-50, then a trend can be determined that can relate to patient A's body mass (a proxy for eating and exercise habits). Then an algorithm can be used to project what patient A's parameters will look like at age 60 or beyond, and/or take the current values for patient A and relate them to the values from other people from a database who had similar scores and project them, e.g. use the available data to calculate the rate of increase in blood pressure given the body mass. This projected result can then be displayed against the observed outcomes from the medical research or articles in several ways.

This is a powerful tool to help physicians persuade patients to (a) change life style(b) take medicine, etc. XBRL gives a clinical physician the ability to develop this data over time and to have periodic analyses and display of the predicted outcome from the treatment, given the data to date.

A secondary use for an embodiment of the present system is to scrub data in medical records to remove a patient's identification and demographic data from the EMR. This type of anonymized data is often sold commercially as part of a multi-million dollar business. Truly anonymized personal health information makes it impossible to link individuals with their data. The data may be used by researchers and public health officials as well as drug companies for their research.

This de-identified data is invaluable for its legitimate public health and research. And, under the Health Insurance Portability and Accountability Act of 1996 (HIPAA) and regulations there is a specification for the elimination of all identifiers as enumerated under HIPAA for its safe-harbor provision. This involves the removal of a patient's name, medical record number, social security number and other data fields that directly link a patient's name.

An exemplary system for implementing the overall system or portions of the invention include a general purpose computing device in the form of a computer, including a processing unit, a system memory, and a system bus that couples various system components including the system memory to the processing unit. The system memory may include read only memory (ROM) and random access memory (RAM). The computer may also include a magnetic hard disk drive for reading from and writing to a magnetic hard disk, a magnetic disk drive for reading from or writing to a removable magnetic disk, and an optical disk drive for reading from or writing to a removable optical disk such as a CD-ROM or other optical media. The drives and their associated machine-readable media provide nonvolatile storage of machine-executable instructions, data structures, program modules and other data for the computer.

Embodiments of the present invention may be practiced in a networked environment using logical connections to one or more remote computers having processors. Logical connections may include a local area network (LAN) and a wide area network (WAN) that are presented here by way of example and not limitation. Such networking environments are commonplace in office-wide or enterprise-wide computer networks, intranets and the Internet and may use a wide variety of different communication protocols. Those skilled in the art will appreciate that such network computing environments will typically encompass many types of computer system configurations, including personal computers, hand-held devices, multi-processor systems, microprocessor-based or programmable consumer electronics, network PCs, minicomputers, mainframe computers, and the like. Embodiments of the invention may also be practiced in distributed computing environments where tasks are performed by local and remote processing devices that are linked (either by hardwired links, wireless links, or by a combination of hardwired or wireless links) through a communications network. In a distributed computing environment, program modules may be located in both local and remote memory storage devices.

Additionally, embodiments within the scope of the present invention include program products comprising machine-readable media for carrying or having machine-executable instructions or data structures stored thereon. Such machine-readable media can be any available media which can be accessed by a general purpose or special purpose computer or other machine with a processor. By way of example, such machine-readable media can comprise RAM, ROM, EPROM, EEPROM, CD-ROM or other optical disk storage, magnetic disk storage or other magnetic storage devices, or any other medium which can be used to carry or store desired program code in the form of machine-executable instructions or data structures and which can be accessed or received by a general purpose or special purpose computer or other machine with a processor. Combinations of the above are also included within the scope of machine-readable media. Machine-executable instructions comprise, for example, instructions and data which cause a general purpose computer, special purpose computer, or special purpose processing machines to perform a certain function or group of functions.

Embodiments of the invention will be described in the general context of method steps which may be implemented in one embodiment by a program product including machine-executable instructions, such as program code, for example in the form of program modules executed by machines in networked environments. Generally, program modules include routines, programs, objects, components, data structures, etc. that perform particular tasks or implement particular abstract data types. Machine-executable instructions, associated data structures, and program modules represent examples of program code for executing steps of the methods disclosed herein. The particular sequence of such executable instructions or associated data structures represent examples of corresponding acts for implementing the functions described in such steps.

In one embodiment, the XBRL-EHR (electronic health record) data is verbally or electronically input to establish, manage, update, transmit and display without reference to other data. A patient's XBRL-EHR is composed of data that relates to the patients identifier or demographic data; e.g., a unique identifier like a Social Security number, date of birth, current address, etc. A patient's XBRL-EHR also contains data relating to the attending physician's clinical observation or interview of the patient, plus the results of laboratory tests, and electronic PACS. The clinical observation of the physician may also be a related to a laboratory result or the reading of a PACS on a certain date; e.g. “on January 1, 2007, neither the MRI nor CT showed abnormalities in the stomach.” Prescriptions for medications are a part of the XBRL-EHR.

The information about the patient is from a variety of sources; interviews, previous medical or official records, laboratory and PACS results, attending nurse records of medications initiated or the patient's response to them, etc. The XBRL-EHR is established by the input of all this data, typically the demographic data as a patient's identifier and a record identifier for the computer. The demographic data is typically input by keypunching, but can be verbally recited with voice translation software, e.g. Dragon Naturally Speaking or comparable, can translate the human voice into data for the XBRL-EHR. Input data may be from paper print outs of laboratory results, which in turn are keypunched or read into translation software to be added to the XBRL-EHR. Similarly, laboratory results, PACS, and prior medical records can be transferred in electronic digital form and used to update the patient's XBRL-EHR.

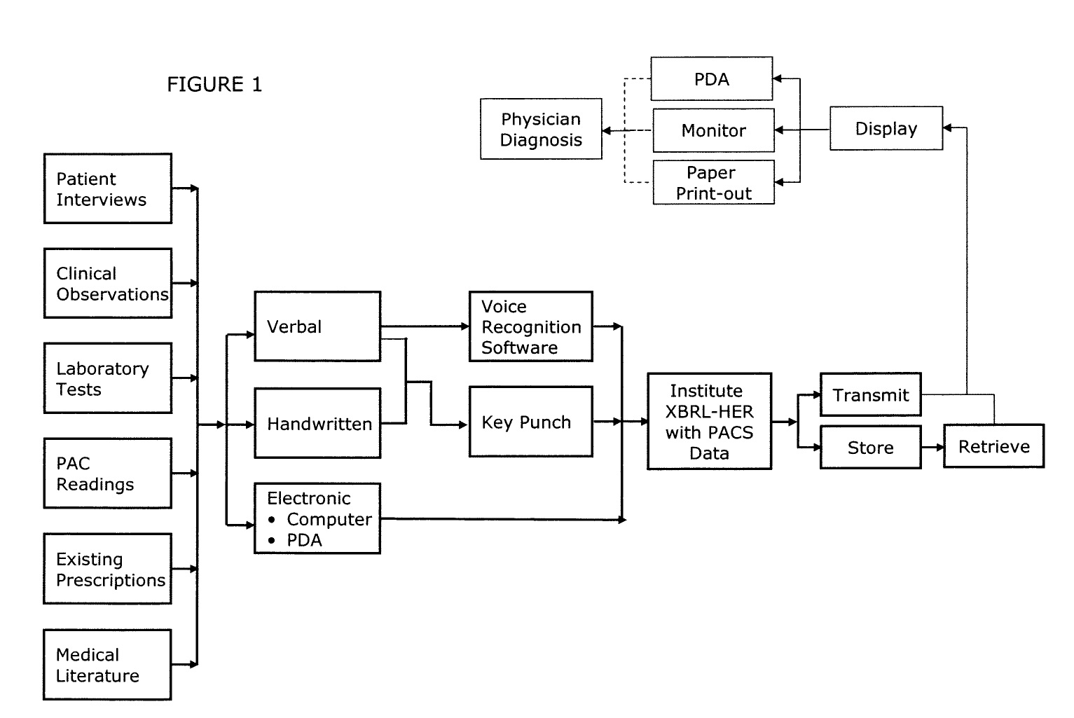

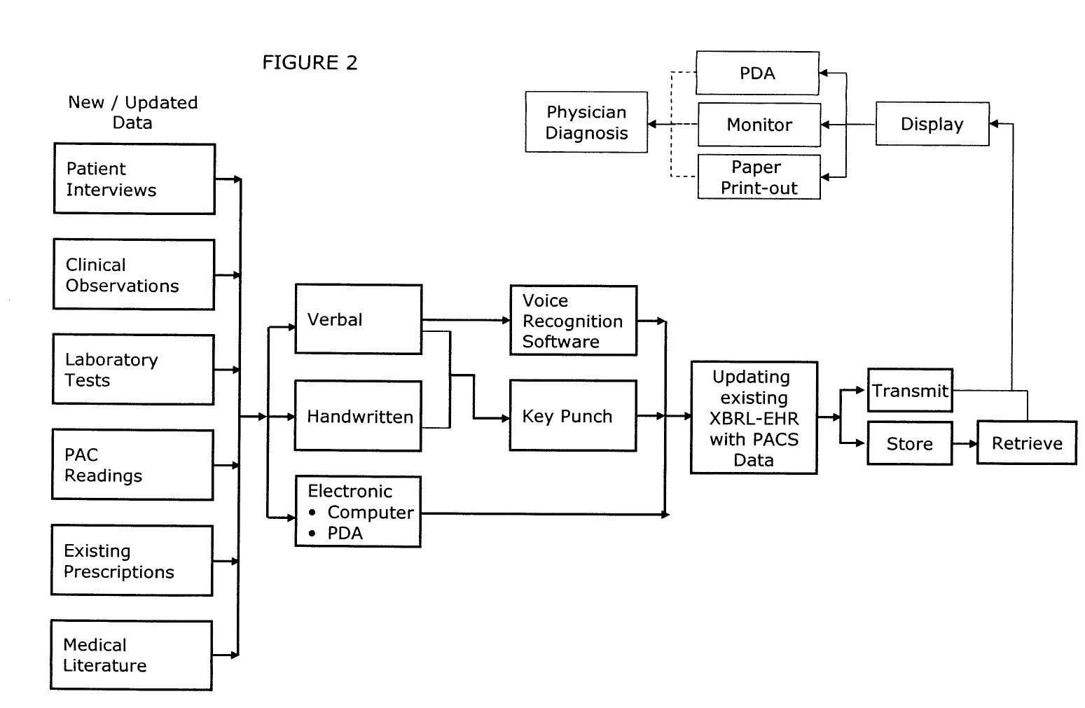

Once the initial patient XBRL-EHR is established, it can be transmitted electronically to an API or computer for storage and retrieved on request or displayed periodically on a scheduled basis. All of the information in the XBRL-EHR can be updated at any time using verbal dictation for software translation into digital electronic records, keypunching, or electronic transmissions. An electronic transmission would include a wireless device the physician or other health care worker used to dictate patient information that is translated into a digital transmission. It would also cover a PDA that might transmit a txt message or e-mail transmission. The updated XBRL-EHR can be transmitted, stored, and retrieved for transmission and display. The display may be on a computer screen, paper printout, PDA or other similar device. (FIG. 1) (FIG. 2)

In another embodiment, the XBRL-EHR data is transferred, in whole or in part, from the originating health organization (hospital, clinic or Regional Health Information Organization) over the Internet to another independent health care organization (hospital, clinic or Regional Health Information Organization) that uses a different unrelated technology platform (computer servers, systems, network, and software). The XBRL-EHR can also be transferred, in whole or in part, to another health organization with an independent computer system and software using the Internet to provide complete transfer, access to, or partial access to the updated XBRL-EHR. Thus, if a primary care physician in Tampa, Fla., referred a patient to a specialist in Atlanta, Ga., the patient's XBRL-EHR could be: (a) transferred electronically to the specialist in Atlanta; (b) copied and a copy electronically sent to the specialist in Atlanta; (c) the specialist in Atlanta could be given electronic access to the patient's record on the primary care physician's computer platform, and the specialist could read it, extract data, perform mathematical diagnostics on it, or, if permitted, update it from Atlanta using the Internet.

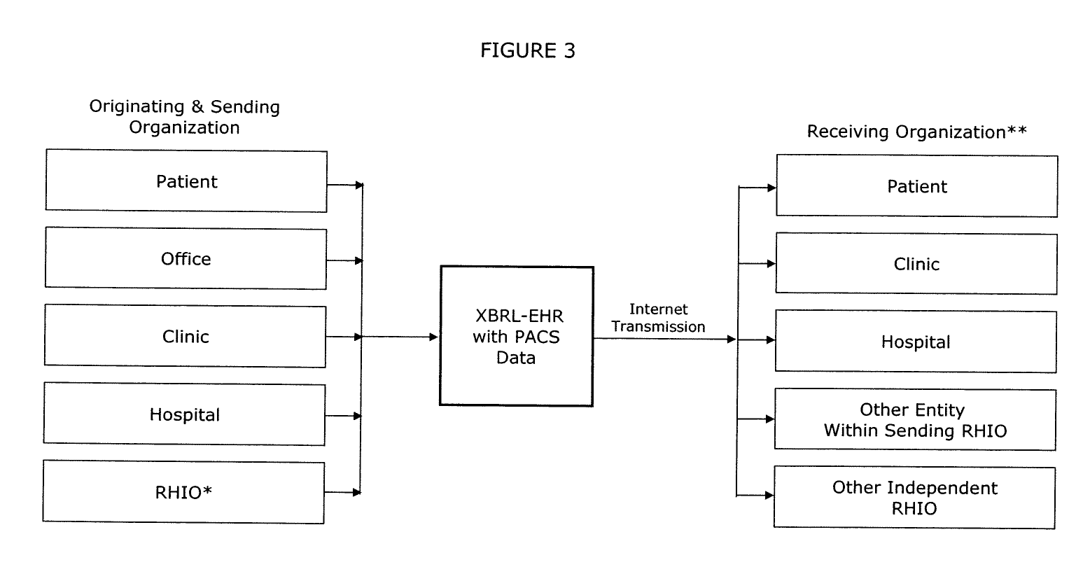

Today, when a patient is referred to a specialist, moved geographically or from one health organization to another their records are paper and if sent, must be copied and typically sent by UPS or FedEx to the new health organization. Even though the PHR paper print-outs sent to the receiving organization is itself a computer print out of an electronic medical record in the sending health organization, this data is often key punched again to be entered into the computer system of the receiving health organization. Typically transferred medical records are printed, copied, and sent by messenger to the receiving health care organization where the information is again key-typed into the computer of the receiving organization. (FIG. 3)

In another embodiment, the XBLR-EHR data is accessed and analyzed using a single or multi-variant mathematical regression analysis to provide longitudinal trends in a patient's clinical, laboratory, or electronic test data. Trends over time provide a physician with important information over time about a patient's current and projected health. Simple measures such as weight, blood pressure, sugar level, cholesterol levels and composition (HDL/LDL ratio), Prostate Specific Antigen (PSA) levels and alike are measures of health or predictors of possible disease. Laboratory blood work that indicates the level of white blood cell count track infections.

This embodiment is demonstrated by the use of regression analysis to calculate longitudinal data about PSA levels. Monitoring software can query the XBRL-EHR data fields that contain the PSA test results from multi-period observations, e.g., 1999, 2001, 2003, 2004, and 2007. Querying the XBRL-EHR, the software digitally receives the patient's current and previous test levels for PSA levels (ng/mL) in blood serum. The software calculates a standard or Cox regression analysis of the PSA rate of change or velocity of the PSA, including the r 2 coefficient of the regression. That is, for each 0.1 ng/mL change in PSA the software calculates the change in the medical hazard ratio from the observations and calculates a Confidence Interval and other dispersion measures such as P values. The software queries a data base of previous PSA test results input from medical literature using the internet, and reported outcomes. A sensitivity and specificity for PSA and PSA velocity are calculated using a receiver-operating curve analysis for PSA values less than some pre-determined levels, e.g. 4.0 ng/mL and 2.6 ng/mL.

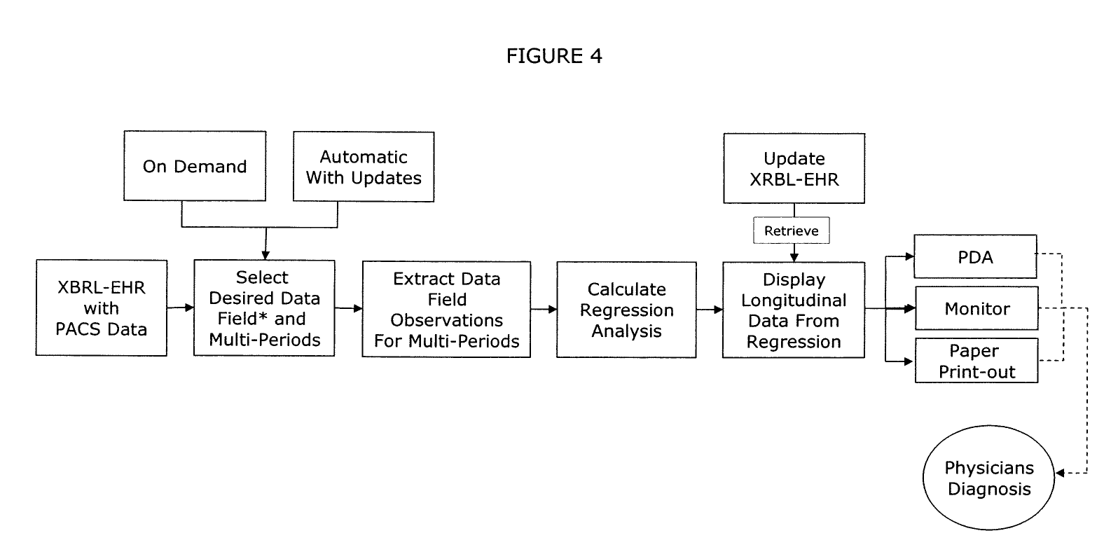

This data is queried from existing databases to demonstrate the patient's velocity results in the context of state of the art using Cox proportional hazards regression, and Kaplan-Meier analysis or other available analyses. These can be transmitted digitally to a remote location and displayed in tabular fashion or displayed graphically on a computer screen, printed, or stored on a computer server for future transmission and display. (FIG. 4)

In another embodiment, the XBRL-EHR data is verbally or electronically input to establish, managed, update, transmit and be displayed with reference to other data from the same organizations computer data bases, or third-party application provider interfaces “APIs” and mashed-up from the Internet. These combined displays or IT mash-ups of data provide a context to view or compare individual observations of clinical, laboratory, or electronic tests (x-ray, CT scans, EKG, etc.) or the longitudinal data (regression analysis) to a selected cohort or reference group, e.g., underweight women over 50 who smoke.

Continuing with the example of PSA test score regression contained in the previous embodiment, this embodiment places that analysis into a context or framework of reference for the physician and patient to better understand the analysis relative to the observed experience of similar peer groups. After calculating the PSA velocity from recent tests, the software will query existing registries or data bases of all relevant medical literature from around the world concerning prostate cancer and benign prostatic hyperplasia to provide a context for the physician and patient to view the test results in the context of the global knowledge and calculate the probabilities and confidence levels of the possible outcomes from the test result.

The software would automatically query and collect all of the known prostate related test data from around the world. It would adjust the data to reflect any national differences or known differences in lifestyles; e. g., Spain has a high proportion of smokers and Japan a diet heavy in fish and limited in red meat that produce hormones often associated with cancers.

From the available global data, which is automatically updated, the software would calculate the test profile of the men who most resembled the patient for comparisons, e.g., black race, smoker, high body mass. This “most similar profile” and its data on survival etc. would be compared with the group of men in the literature as a whole. The patient's test results could be compared to the “most similar profile” and the “profile” of the group as a whole. Both test results could be transmitted, displayed, printed or stored for future use.

While the data about differences in race and lifestyle are currently limited, by attempting to collect them and factoring them into the software's database, they would become more statistically meaningful. The software would accept data on other men tested for PSA and PSA velocity, their lifestyle profile for future comparisons by the software or as a database available for future medical research. The patient's test and profile data could be entered into an updated database and combined with the men's database as a whole and by race and lifestyle to determine whether there are statistically significant differences in predicted outcomes, confidence levels, and other statistical measures.

The software uses these calculated velocity values from the patient's current and past test results (ng/mL) and relates them to the available population of past data and analysis of PSA levels, velocity, and age. It then calculates the probabilities of outcomes and confidence levels relating to the patient's test data and available medical research about the population in general or the specific patient's race, body mass index, etc. That is, the software will calculate the patient's PSA volatility, level and volatility, age and plot the data over time and display it graphically against the existing medical research. This can provide the probability of the patient never contracting prostate cancer, the probability that the patient may contract prostate cancer, but that it may not be treated given the patient's age, and the probability of it occurring and its severity. Severity is measured by the conventional Gleason score based on past observations.

The software has a query capability so that the user may query the database for custom regression analysis, and access medical journals and statistical references to provide context and treatment options to the user. For example, under each treatment heading; radiation, chemotherapy, and surgery the medical literature can be accessed and displayed or printed for the patient. This literature database as well as the PSA database is continually updated to provide the latest treatment options and statistical projections of PSA test levels and velocity given the patient's age. A number of search appliances will be used to search inside and outside the software's data (within the enterprise and outside the enterprise). For example, Google, Yahoo, Thunderstone, and Powerset will be incorporated into the software for Internet and enterprise query capabilities.

One example of the data from outside the enterprise is the number of prostate surgeries performed by a particular hospital in a selected geographic location, and where available a particular doctor. Similarly, the reported incidences of impotence and incontinence by surgeon or hospital will be reported when available.

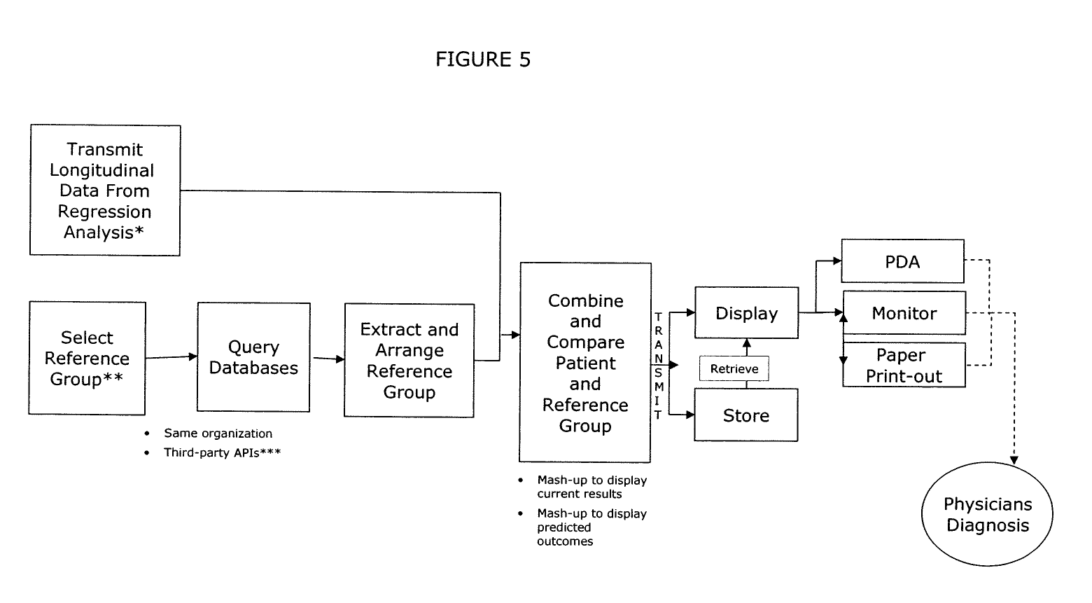

This embodiment takes the calculated regression analysis or other mathematical calculation of the patient's data, e.g., a distribution diagram and places them in context to assist the physician and patient in understanding test results. Drawing on other public or private data from other API's the software can mash-up the patient's test data with the available global data to provide context for the test results. XBRL-EHR enables this type of diagnostic software to operate. (FIG. 5)

In another embodiment, the XBLR-EHR patient identifying data is “scrubbed” or blocked to remove certain designated demographic or identifying data from an XBLR-EHR, and the resulting de-identified XBLR-EHR is transmitted) over the Internet to an independent data management service bureau or health care organization that will use the de-identified XBLR-EHR for non-diagnostic public health, medical, or pharmaceutical research purposes. The software has the capability to prohibit the copying or transmitting of certain data fields containing the patient's identify or identifying demographic data from being copied or transmitted as secondary public health or research purposes.

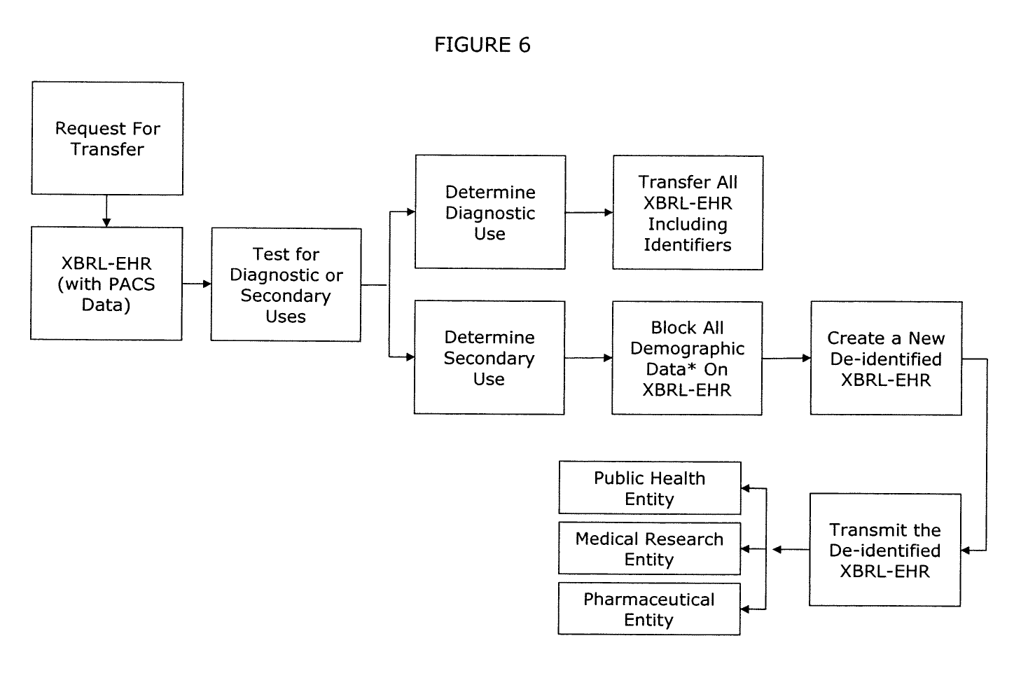

Prior to copying, aggregating, or transmission, the software will determine the use of the XBRL-EHR. When the use of the XBRL-EHR is identified as public health or research, then the XBLR-EHR is “scrubbed” or the identifying or demographic data blocked on the originating health organizations computer system prior to the clinical data being copied and transmitted either to an intermediary data service bureau or directly to the end user for secondary purposes. The software identifies the use of the data in each instance and when it is to be used for secondary public health or research purposes, it de-identifies the XBRL-EHR and blocks the transmission of the identifying demographic data. It does this by identifying the data fields for all the identifiers in the personal health record and suppressing or not permitting them to be viewed for secondary data use purposes. This is consistent with the report “Toward a National Framework for the secondary Use of Health Data” by the American Medical Informatics Association, Sep. 14, 2006. See: www.amia.org. (FIG. 6)

In another embodiment, the XBRL-EHR information is used to initiate, validate the claim, and pay the patient's health insurance claims. A health care provider can use a XBRL-EHR to interoperate with any computer program that the health care provider to any health care payors computer platform. As long as both parties have access to the Internet the software can extract a patient's identification and demographic information from the XBRL-EHR records, while XBRL software interacts with any computer program of a payor to extract the payor's form to initiate the payment claims process. Using a universal identifier such as a Social Security number, the XBRL enabled software will complete or fill in the payor's form that initiates the payment process. This payment process may be a claims process for a private sector health insurer, a military paying agency, or other government entity regardless of the software they use.

Once the form to initiate payment is electronically completed, it is next evaluated. The software can evaluate or test the form against predetermined criteria to ascertain specific factors, e.g., co-payment obligations of the patient. The evaluated form to initiate payment can be electronically transferred to predetermined computer monitors for display and/or stored on the web for future access by authorized individuals who inquire about the payment.

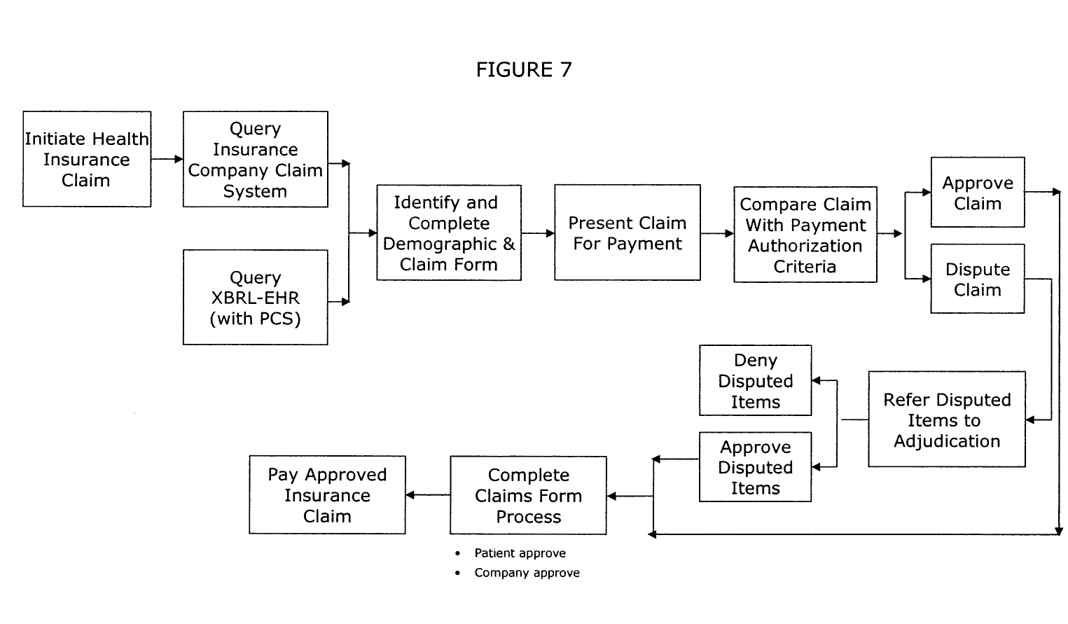

The evaluated form to initiate payment can be tested for authorization for payment or rejected for payment pending further analysis by a human being. If authorized, the approved form to initiate payment can be electronically transmitted to a computer program that will either initiate a check authorization and printing process or an electronic payment to the patient. The result of the authorization of payment can be transmitted to the appropriate audit officials and displayed electronically. If unauthorized for payment, the form to initiate payment can be electronically transmitted to predetermined computer monitors or remain stored on the web subject to being requested and electronically displayed for the relevant human review. Alternatively, a form to request payment can also be compared against other specific criteria that permit its analysis against predetermined criteria that the form to request payment is subject to a secondary electronic review or human review. In either case it is transmitted and the results of a second electronic review are displayed on a computer monitor. In the case of a human review the results can be electronically transmitted to predetermined computers or stored on the web for future computer display and human analysis. (FIG. 7)

In one embodiment, software and systems utilize XBRL to transfer a patient's medical records from the physician's office, clinic, hospital, medical laboratory originating the record (or having the updated current record), to another location (physician's office, clinic, hospital or payers). This transfer is done by electronically transferring the patient's health records in their native computer format to a website that uses XBRL to make the electronically received data interoperable. In another embodiment, the transfer is made by electronically transferring the patient's health records in their native computer format using XBRL software to make the electronically received data interoperable, that is, to enable the ability to be received by another computer that is not in the native computer language or data standard different from the native computer language or data standard of the entity originating or updating the patient's record.

Depending on operator instructions, the website immediately forwards the medical record to another location or maintains it on a server for future access. Access and/or transfer at a future time is made by electronically linking the requesting party to the website and displaying, transferring, or printing the records. These transfers may be using XBRL or other types of software appropriate to multi-media or large scale data storage and transfer. Appropriate encryption and password protections are used to ensure the privacy and security of the patient's medical health care record. For medical research purposes, the website can remove an individual patient's identification and aggregate the patient's data using pre-determined criteria for the medical research organization. This is called “de-identification.”

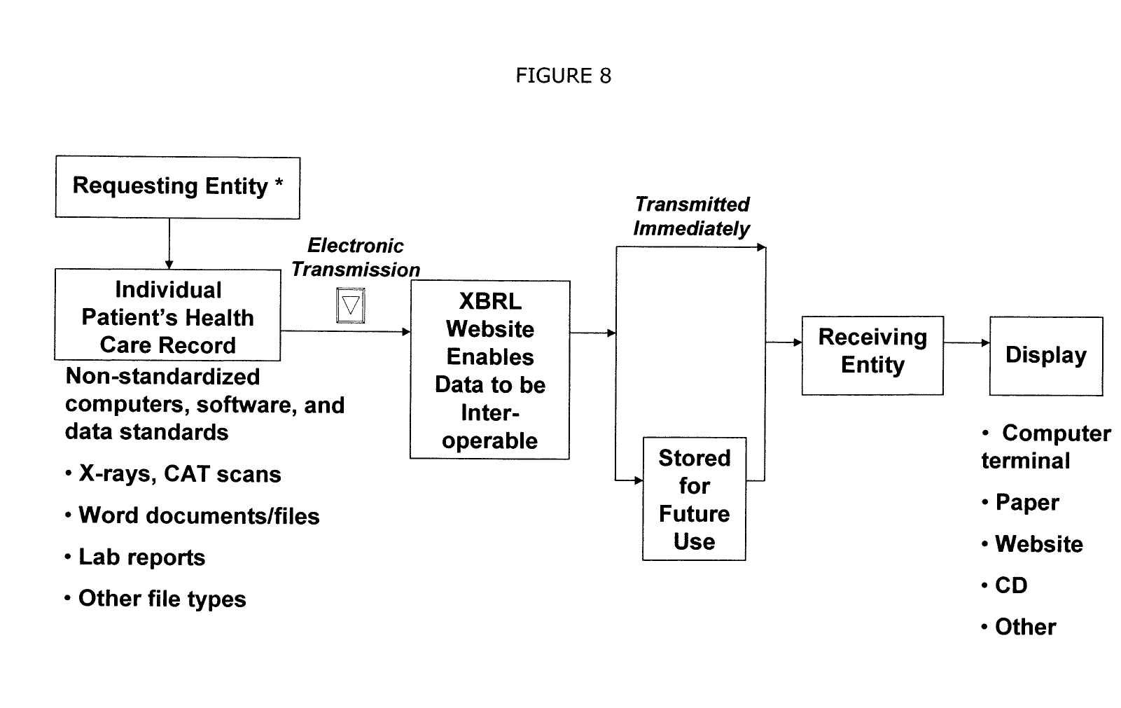

In one embodiment, the patient requests the originating physician's office to transfer the patient's medical health care records to another physician for consultation. In this case, the originating physician provides the necessary authentication codes to the website and authorizes the transfer of the patient's medical health care records to the receiving physician's location. The records, including a variety of multi-media file types and x-ray, CAT scans, mammograms, and other diagnostic data are included in the patient's health care record that is transmitted to the website. The receiving location can store electronically, display, or print the patient's health care record for viewing or analysis of the underlying data, e.g., for genotypic data analysis. (FIG. 8)

In another embodiment, the originating physician's office initiates the transfer of the patient's health care records to another physician for consultation. In this case, the originating physician provides the necessary authentication codes to the website and authorizes the transfer of the patient's medical health care records to the receiving physician's location. The records are multi-media including a variety of file types such as x-rays, CAT scans, mammograms, and other diagnostic data are included in the patient's health care record that is transmitted to the website. The receiving location can store electronically, display, or print the patient's health care record for viewing or analysis of the underlying data, e.g. for genotypic data analysis.

In another embodiment, a patient's health care payer initiates the transfer of the patient's health care records to another physician for consultation or to the payer or their agent, e.g., lawyer. In this case, the payer provides the necessary authentication codes to the website and authorizes the transfer of the patient's medical health care records to the receiving physician, payer, or their agent's location. The records, including a variety of file types such as x-rays, CAT scans, mammograms, and other diagnostic data are included in the patient's health care record that is transmitted to the website. The receiving location can store electronically, display, or print the patient's health care record for viewing or analysis of the underlying data, e.g., for known allergies or current medications.

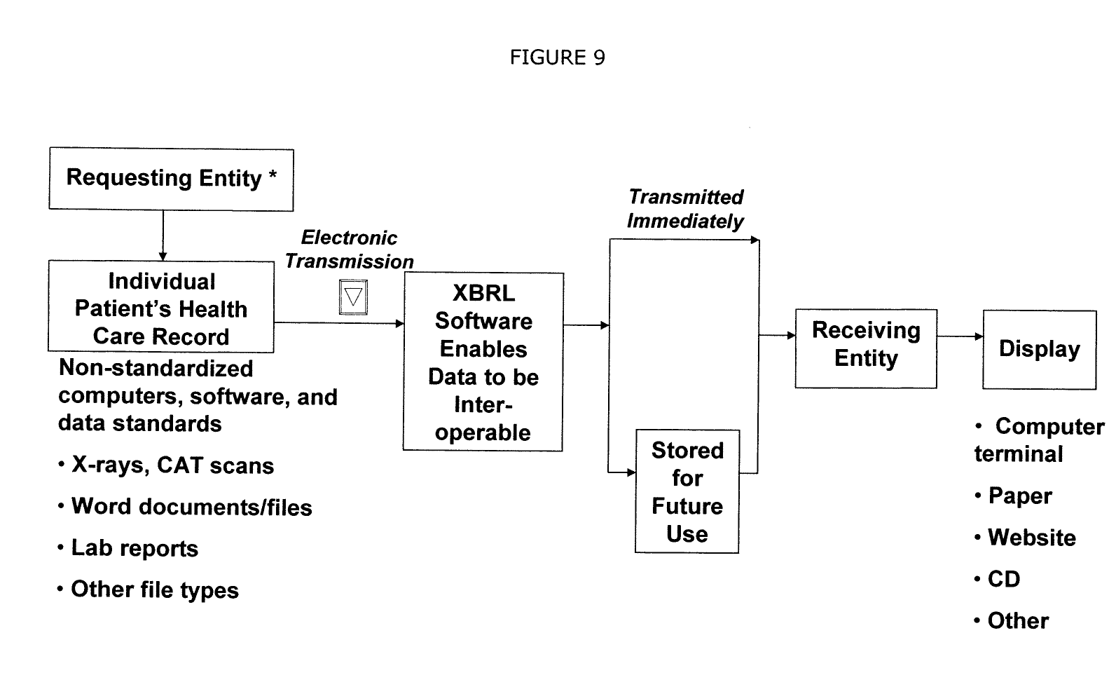

In another embodiment, the patient, originating physician or other authorized entity initiates the transfer of the patient's health care records to another receiving entity. The records, including a variety of file types such as x-rays, CAT scans, mammograms, and other diagnostic data are included in the patient's health care record that is transmitted to the receiving entity utilizing software that is XBRL enabled. The XBRL software will employ an XBRL enabler and translator. The receiving location can store electronically, display, or print the patient's health care record for viewing or analysis of the underlying health care data, e.g., for current prescriptions. (FIG. 9)

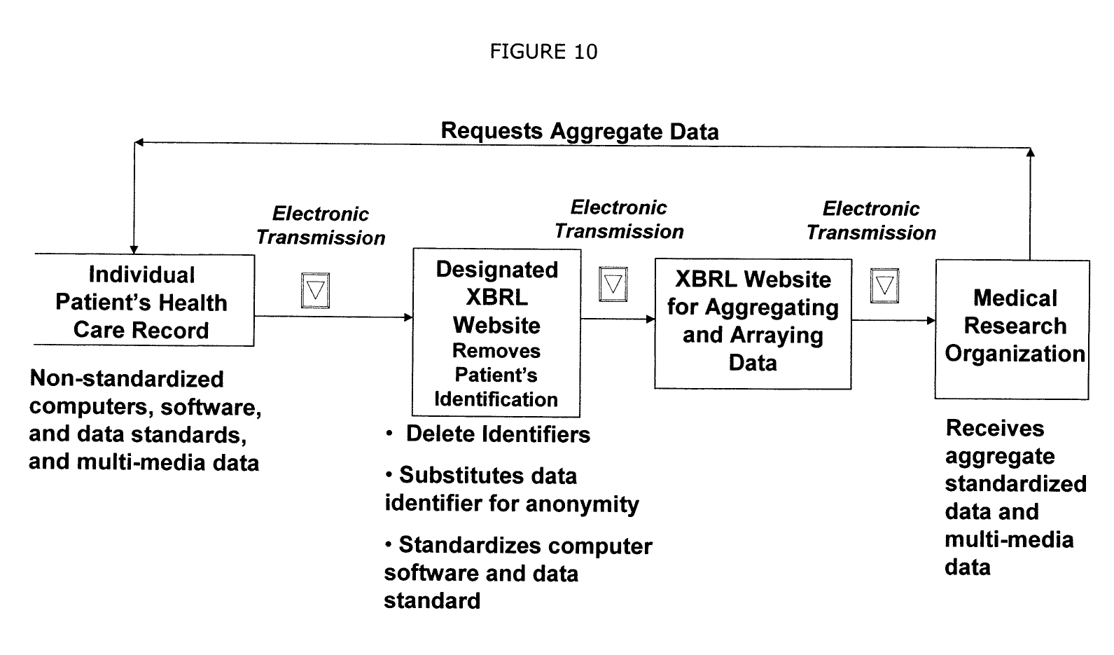

In another embodiment, a medical research organization wishes to acquire aggregate data on a class or type of patients without identifying the individual patients by name, but rather collecting statistically significant data by some pre-determined criteria. Such criteria might include, but are not limited to, geographic birth location of the patient, geographic current residence of patient, age, sex, race, ethnic group, smoking or non-smoking, height to weight ratio, and alike. In this case, the originating medical research organization provides the necessary authentication codes to the website and authorizes the transfer of the patient's medical health care records without patient identifiers to a designated Internet address and file format for analysis. The individual records are then searched and arrayed in the format desired, e.g., “men over 65 who smoke.” This searching may be by Boolean algebra, Google or ask.com technology, or metadata analysis. The individual patient records, including a variety of file types and x-rays, CAT scans, mammograms, and other diagnostic data are included in the aggregate encrypted data that is transmitted to the designated Internet address and file format. The receiving Internet address and file format can store electronically, display, or print aggregate patient's health care records for viewing or analysis of the underlying data, e.g., for genotypic data analysis. In aggregating encrypted individual patient data, but not individual patient identities, unique identifiers may be used that include alphanumeric and metadata coded identities. (FIG. 10)

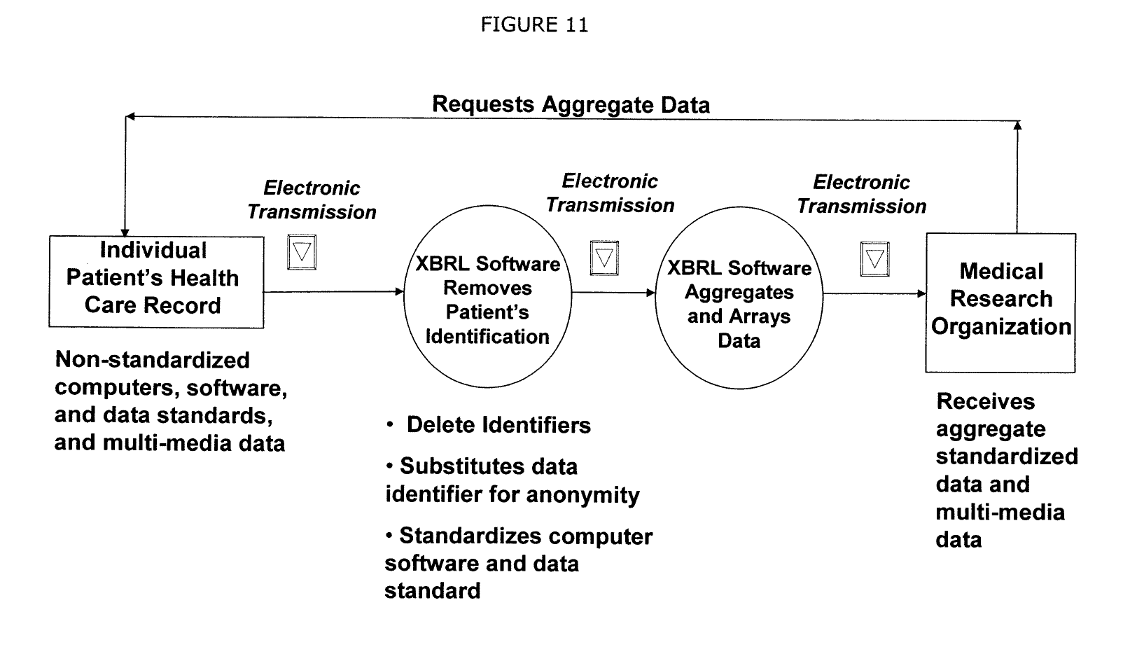

In another embodiment, a medical research organization wishes to acquire aggregate data on a class or type of patients without identifying the individual patient by name, but rather collecting statistically significant data by some pre-determined criteria. Such criteria might include, but are not limited to, geographic birth location of the patient, geographic current residence of patient, age, sex, race, ethnic group, smoking or non-smoking, height to weight ratio, and alike. In this case, the originating medical research organization provides the necessary authentication codes and the XBRL software enables the transfer of the patient's medical health care records without patient identifiers to a designated Internet address and file format for analysis. (That is the software does it without a website as in the previous embodiment.) The individual records are then searched and arrayed in the format desired, e.g., “men over 65 who smoke.” This searching may be by Boolean algebra, Google or ask.com technology, or metadata analysis. The individual patient records, including a variety of file types such as x-rays, CAT scans, mammograms, and other diagnostic data are included in the aggregate encrypted data that is transmitted to designated Internet address and file format. The receiving Internet address and file format can store electronically, display, or print aggregate patient's health care records for viewing or analysis of the underlying data, e.g., for genotypic data analysis. In aggregating encrypted individual patent data, but not individual patient identities, unique identifiers may be used that include alpha-numeric and metadata coded identities. (FIG. 11)

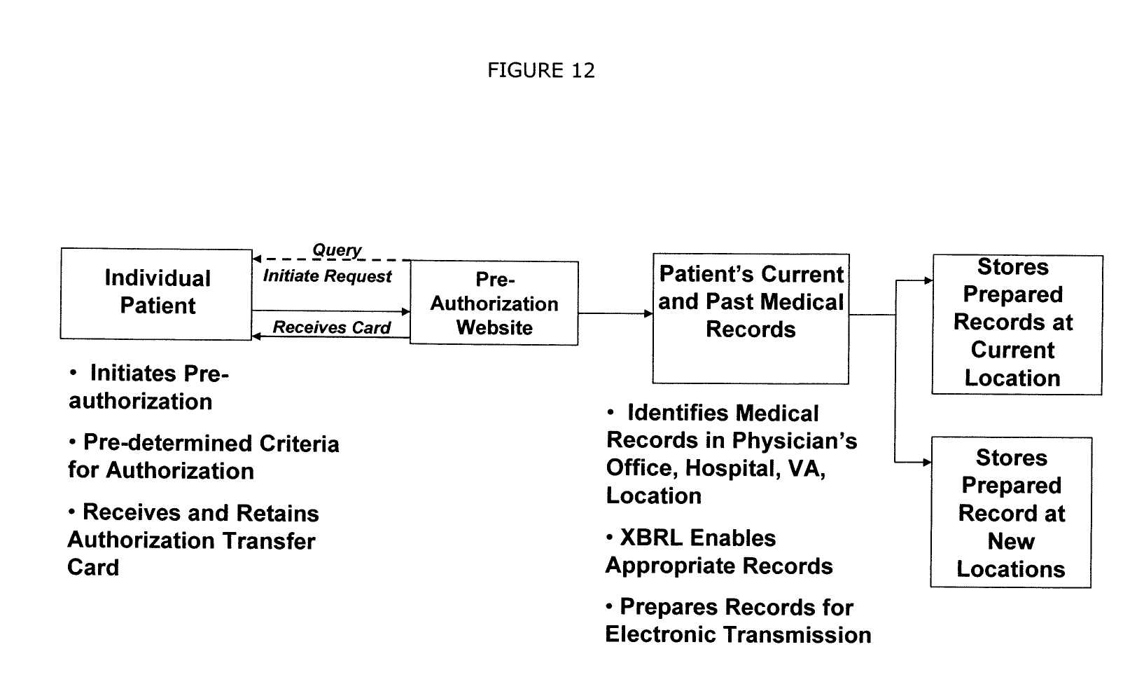

In another embodiment, the receiving party may also be the party that originates the request for the patient's medical record. Here, the receiving party may query the patient's health record remotely over the Internet. This is the same Internet query technology used to check users of computer products to provide them with updates to the products, e.g. an HP 6210 Office Jet All in One owner receives product updates over the Internet. Similarly, a computer virus protection service such as Norton Anti-Virus can remotely inquire and determine whether their software requires updating and do so over the Internet. Using these types of remote inquiries and commands, the invention can enable someone other than the patient to initiate the inquiry for the patient's medical records. The patient can either subscribe to a service and authorize the transfer in advance or simply respond to an e-mail on their computer that authorizes the party initiating the transmission to do so. Security features will be built in to prevent the transmission of the individual's health care record to an unauthorized entity. Similarly, the patient's unique identifiers will be removed in the case of a research organization seeking aggregate data. (FIG. 12)

In another embodiment, a web site service is established that people can use to pre-authorize the electronic transfer of their individual medical records. In turn, the individual patient receives and retains a card with authorizing encrypted codes indicating to the web site or other authorized receiver that the records can be transferred at the time or by a physician who determines that the patient, who may be unconscious, has pre-authorized the electronic transfer. This is similar to an organ donor giving their consent to the use of their organs after death. This pre-authorization of the individual's medical records provides the legal, privacy, security and HIPPA authorizations for the attending physician to use the Internet to obtain immediately the patient's electronic health care records in a medical emergency. The individual patient can pre-authorize or consent to the transfer on the website and the subsequently attending physician could determine whether the patient had given their pre-authorization or not on the website. The physician could then request the patient's medical records and use them in their diagnosis and treatment of the patient or proceed with the diagnosis and treatment without the advantage of the patient's medical records.

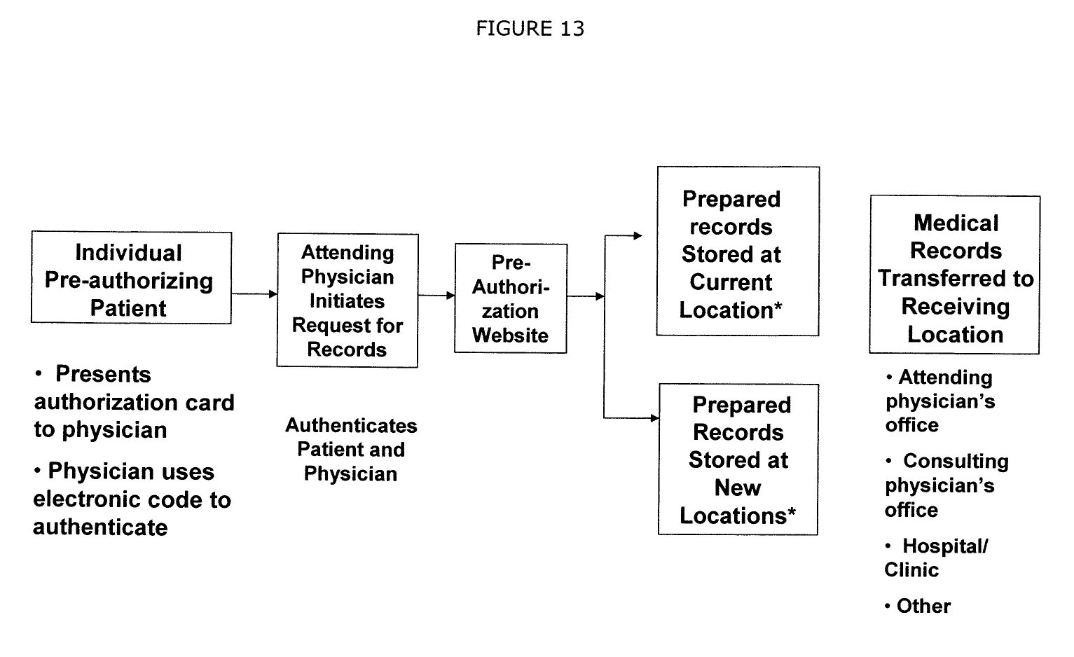

In the event that some of the patient's individual medical records are not transferable from an information technology standpoint, they may be immediately accessed by the requesting physician and viewed over the Internet from the website to complete the attending physician's diagnosis of the patient. For example, multi-media records such as x-rays may be retained at the location, but accessed remotely for viewing. (FIG. 13)

The invention is web-based XBRL enhanced XML application software that is interoperable between health care provider and health care payor computer systems and provides them functionality that they do not have without being interoperable. Utilizing the XML/XBRL interoperability feature, over the web, the software will automatically utilize information from a patient's health care record and the computer software of the payor to work on an interoperable basis.

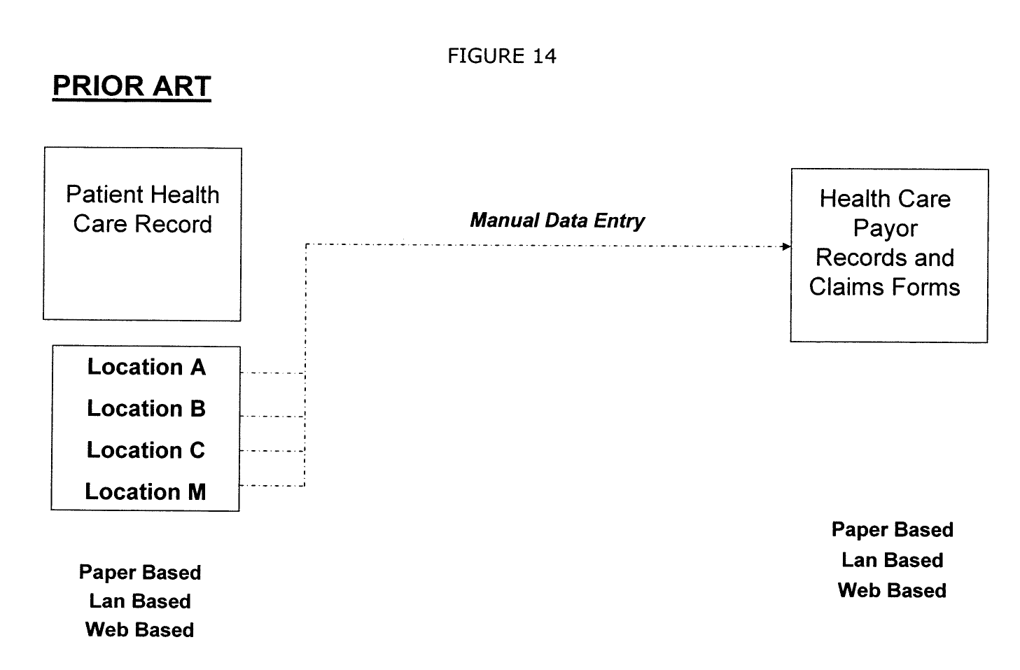

At the simplest level, using just the patient's unique national identifier, e.g. Social Security number, the software utilizes XBRL's interoperability capacity to read the identified patient's personal information and medical record from any computer software that the health care provider uses. It then reads the health care payor's insurance claim form, regardless of the payor's software, completes the insurance claim form on line and enables the user to conduct analysis of the data, manipulate the information, process it, transmit it, and display the results on any computer monitor that has access to the web. Hence the computer monitor can be on another floor of the health car provider or in another location on the globe. (FIG. 14)

For example, if a patient's Social Security number in the patient's medical record (XBRL instance document) were used to fill out the patient's insurance form (XBRL taxonomy) electronically and produce an electronic document that was an insurance claim form (instance document), it can be analyzed, transmitted, and displayed remotely. The combining of these two functions increases information accuracy, saves processing costs,

Without the interoperability of patient health care records and the health care payors' records, there are key punch errors, repetitive data validation requirements, HIPPA privacy concerns about patient's records, and added costs. Today a patient often does not have all of the required information available at the time of checkout to effectively check out of a health care hospital or clinic. This results in lost time, delays, and added cost to the health care system. The lack of interoperability of patient and payor computer systems impedes analysis of patients and payors activities for quality control, audit, and health insurance underwriting analysis. Combining the data from the two systems permits automatic electronic analysis of data without costly hand keypunching and other errors.

A health care provider can use XBRL enabled XML software to interoperate with any computer program that the health care provider has and extract a patients health and personal information from those records, while XBRL software virtually simultaneously interacts with any computer program of a payor to extract the payor's form to initiate the payment claims process. Using a universal identifier such as a Social Security number, the XBRL enabled software will complete or fill in the payor's form that initiates the payment process. This payment process may be a claims process for a private sector health insurer, a military paying agency, or other government entity.

Once the form to initiate payment is electronically completed, it is next evaluated. The software can evaluate or test the form against predetermined criteria to determine specific factors, e.g. co-payment obligations of the patient. The evaluated form to initiate payment can be electronically transferred to predetermined computer monitors for display and/or stored on the web for future access by authorized individuals who inquire about the payment.

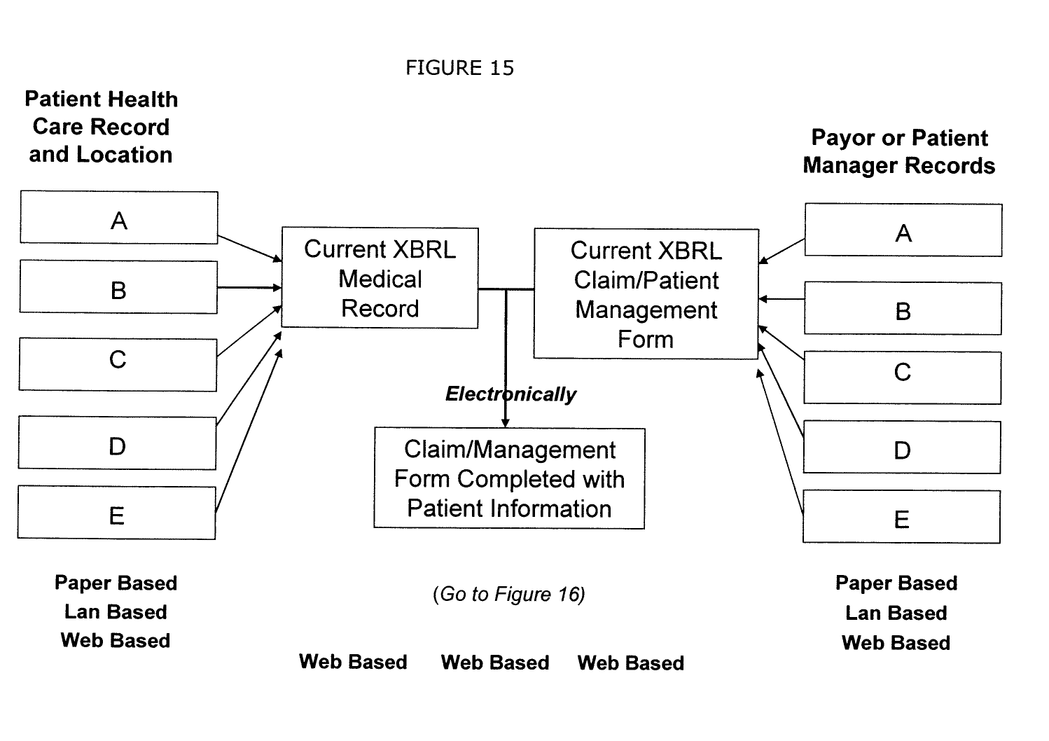

The evaluated form to initiate payment can be tested for authorization for payment or rejected for payment pending further analysis by a human being. If authorized, the approved form to initiate payment can be electronically transmitted to a computer program that will either initiate a check authorization and printing process or an electronic payment to the patient. The result of the authorization of payment can be transmitted to the appropriate audit officials and displayed electronically. If unauthorized for payment, the form to initiate payment can be electronically transmitted to predetermined computer monitors or remain stored on the web subject to being requested and electronically displayed for the relevant human review. Alternatively, a form to request payment can also be compared against other specific criteria that permit its analysis against predetermined criteria that the form to request payment is subject to a secondary electronic review or human review. In either case it is transmitted and the results of a second electronic review are displayed on a computer monitor. In the case of a human review the results can be electronically transmitted to predetermined computers or stored on the web for future computer display and human analysis. (FIG. 15)

The invention is web-based XBRL enhanced XML application software that is interoperable between the electronic health care record for an individual using their genotype information (“genotypic information”) and existing information sources about the interaction of a pharmacological, chemotherapeutic or radiological treatment on a particular genotype structure found in the individual patient's electronic health record.

The human genome has been mapped in the laboratory workbench, but its genotypic data is not been used by doctors at the patient's bedside. The capability to identify a patient's genotypic data or genotype, which is a reflection of their genomic make up can be used as an early warning for detecting diseases, to tailor or customize treatments including drug prescriptions, protein therapy, or gene therapy for their patients and relate types of treatments to their individual patients, e.g. radiation treatment or chemotherapy. Thus, with present system the medical profession can compare a patient's data, e.g., genotype with pharmacological attributes of a drug to determine the best type of drug and dosage for that individual patient's genotype to prescribe optimum drug and dosage for that individual.

At the simplest level, using just the patient's unique national identifier, e.g. Social Security number, the software utilizes XBRL's interoperability capacity between and among other software systems to read the identified patient's personal information, e.g., genotype from any computer software that the health care provider uses or has access to on the internet. Using XBRL and the medical ontology's available, the software identifies the possible treatments for a disease, and then compares the treatments effect on the patient's genotype from the patient's phenotypic data.

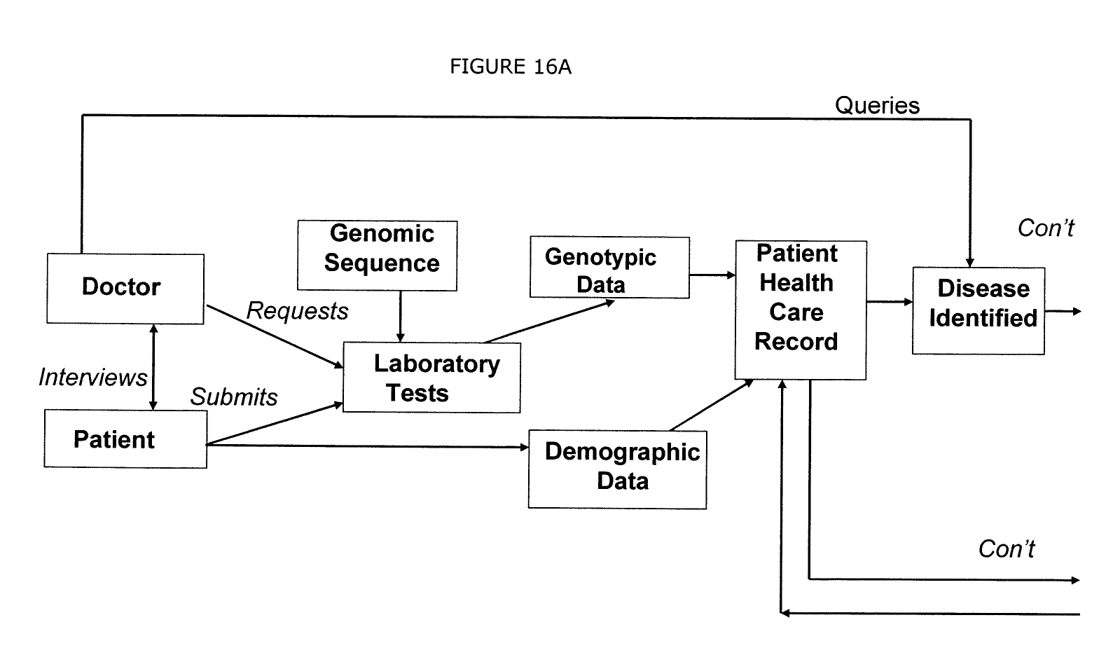

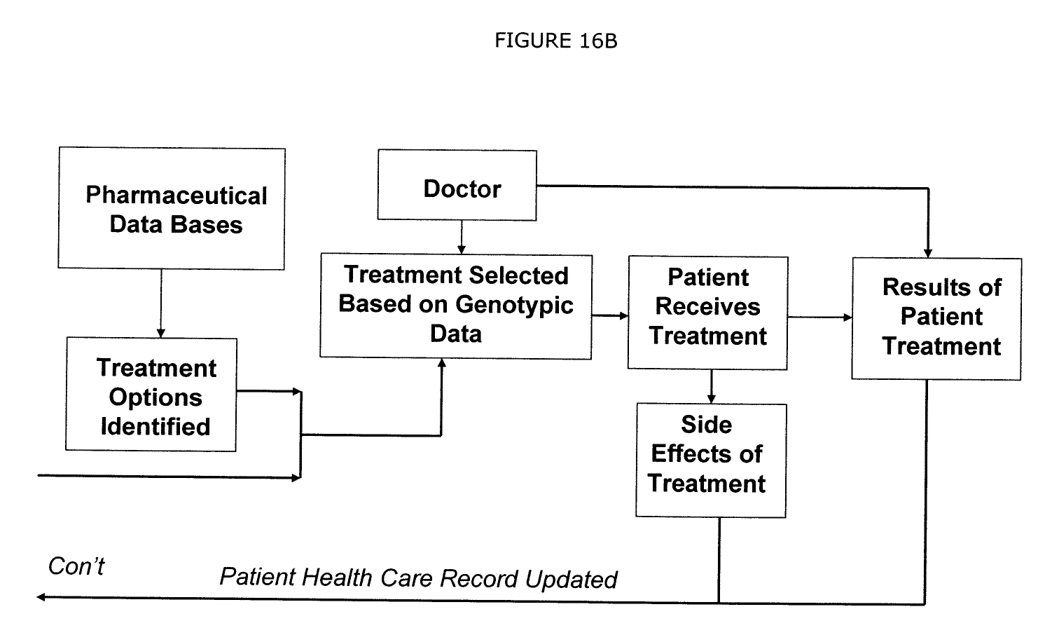

The software can then rank the available treatments by a variety of factors, e.g., most recent, strongest, fastest, safest (least side effects), longest in use, and alike. These results are then displayed in human readable language so that a physician can select the drug or combinations of drugs and/or treatments that will have the optimum prophylactic effect given the individual patient's genotype. This equips the physician to make better judgments about available treatments and associated risks given the knowledge of how the treatment is likely to interact with the patient's genotype. (See FIGS. 16A and 16B)

In one embodiment, the XBRL software can utilize a patient's health care record and then select the patient's genotype from the record. It can then use the internet to identify and compare that patient's genotype with existing genomic data bases to identify the patient's propensity to a particular disease, e.g. diabetes. This is based on the empirical evidence collected to date and the identified probabilities of the individual developing a particular, given their genotype. This data can then be transmitted over the internet to any computer monitor and presented in human readable form or printed in paper form for a human to read.

In another embodiment, the XBRL software can utilize a patient's health care record and then select the patient's genotype from the record. It can then use the internet to identify existing data bases to determine whether the individual is suffering from a particular disease, e.g., diabetes and the severity of the disease at present. It can then search the internet for information indicating the rate of progress the disease is likely to take over some time in the future. The physician can specify the length of time in the future wanted to project the progress of the disease. This data can then be transmitted over the internet to any computer monitor and presented in human readable form or printed in paper form for a human to read.

In another embodiment, the XBRL software can utilize a patient's health care record and then select the patient's genotype from the record. It can then use the internet to identify existing medical data bases to determine whether the individual is suffering from a particular disease, e.g. diabetes and the severity of the disease at present. It can then search the internet for information indicating the rate of progress the disease is likely to take. The software can then use the internet to identify possible remedies to treat the disease, e.g., insulin in the case of diabetes. Then using predetermined criteria, the software can compare the individual patient's health data or genotype requirements for insulin and calculate a treatment program with appropriate dosages and the optimum timing of the dosages to treat the individual patient. The software can then recommend a treatment program or display model treatment programs for the physician to read and evaluate. This data can then be transmitted over the internet to any computer monitor and presented in human readable form or printed in paper form for a human to read. The data can then be stored and used in the future to monitor the individual patient's response to the treatment over time and adjust the quantity of drug and frequency.

In another embodiment, the XBRL software can utilize a patient's health care record and then select the patient's genotypic data from the record. It can then use the internet to identify existing data bases to determine whether the individual is suffering from a particular disease. It can then search the internet for information indicating the rate of progress the disease is likely to take over some time in the future. The software can then use the internet to identify possible remedies to treat the disease, e.g., in the case of high cholesterol, the drug Lipitor (atorvastatin calcium). Using predetermined criteria, the software can then compare the individual patient's cholesterol level and genotypic to calculate a treatment program with appropriate recommended dosages and the optimum timing of the dosages of Lipitor to treat the individual patient. The software can also recommend a treatment program or display model treatment programs for the physician to read and evaluate. This data can then be transmitted over the internet to any computer monitor and presented in human readable form or printed in paper form. The data can then be stored and used in the future to monitor the patient's response to the treatment over time and adjust the quantity of Lipitor, e.g. 10 mg, 20 mg, 40 mg, or 80 mg or their frequency.

In another embodiment, the XBRL software can utilize a patient's genotypic data and current drug usage, e.g. 40 mg. Lipitor for high cholesterol to monitor for developments in the literature concerning reported side-effects from the use of the drug or the introduction of another new drug to replace or supplement the current drug. Using the internet, the software can identify new information and drug trials about the usage and side effects of Lipitor over time. The software can use predetermined criteria to send an electronic alert message to the doctor and/or patient when meaningful new information about the use of Lipitor and its effects become available. The patient's medical record can be updated or linked by XBRL to the new information so that the doctor and the patient can make a re-informed treatment decision about continuing the current dosage, and frequency of the Lipitor treatment given the new information about its efficiency, side effects, etc.

In another embodiment, all individual patients health records can be stripped of their individual or personal identifiers and combined on the internet to provide a regional, national or international data base of the treatment for all patients and their results measured at the genotypic levels and combined with phenotypic information to provide large scale measures of disease treatments and their efficiency. This can be used to assist doctors in prescribing particular medicines, amounts, and frequency of use. The software will provide the means to analyze, compare, and publish the results of this effort by disease, disease stages, geographic subdivision, age, sex, and genotypic characteristics. These results can be tabulated, then transmitted, and displayed in human readable form on a remote computer screen or print out. In turn, this information will be used by doctors to make better informed patient treatment decisions for new patients. This is the genotypic data bank envisioned in the earlier embodiment to provide the data needed for improved initial treatment.

In another embodiment, the patient's genotypic records are used to identify the patient's genes that may be modified to treat the patient's own existing cancer or to modify the genes to prevent a cancer from occurring in the future. For example, a patient's Liposites, may be extracted and reformulated to attack the patient's existing cancer or focused on preventing the development of a cancer that the patient's genotypic data indicates an higher probability of occurring. In simple terms, this is modifying a patient's genes to teach them to fight the patient's existing or prospective cancer and eliminate it or cause it to be in remission or not grow.

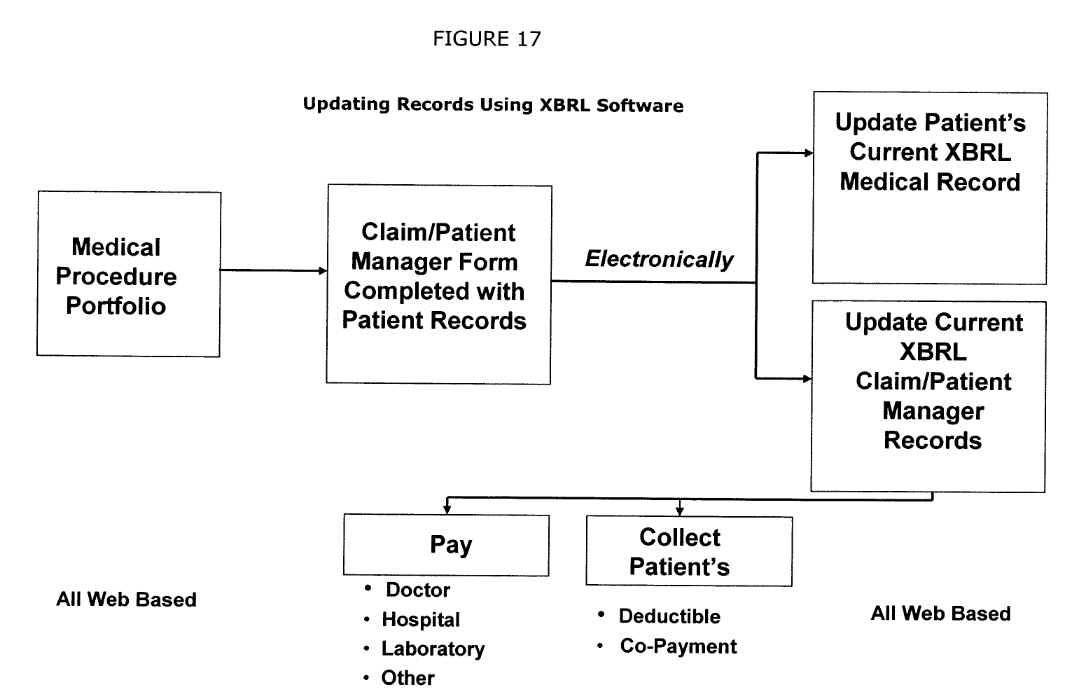

Referring now to FIG. 17, there is illustrated one embodiment of an XBRL healthcare management system. Block 100 comprises an operation of extracting and/or receiving or generating a medical record comprising at least clinical examination data or laboratory results and context data therefor for a patient. In one embodiment, the medical record may be an electronic medical record in an electronic medical database that is accessed and retrieved by means of the Internet or other network such as an extranet or intra-net particular to an organization. The electronic database may be maintained by a hospital, physician, medical insurance payor, or any other healthcare related operation, or a database maintenance company and may include primary or secondary patient medical data on an electronic medical record. Alternatively, a facsimile of a medical record may be received and converted into an electronic medical record. Alternatively, a paper medical record may be received and converted to an electronic medical record. The method by which the medical record is extracted, or received or generated is not limiting on the invention.

Block 102 comprises an operation of creating or updating an XBRL or equivalent medical record in a patient medical repository. The term “repository” means a collection of data that includes XBRL data records, but may also include medical data in its native XML form or in any other convenient format. The XBRL medical record comprises XBRL data fields containing clinical examination data and/or laboratory results made over a period of time and associated metadata representing attributes at the data value level based on a medical taxonomy. This operation comprises in one embodiment mapping the clinical examination data and/or laboratory results into one or more XBRL data fields and creating based on the context data metadata representing attributes at the data field level based on a medical taxonomy and associating the metadata with the data field. The term “associating” is intended to encompass the act of electronically linking. This conversion into an XBRL or equivalent medical record is accomplished by a conversion engine, which operates to take each of a plurality of items of data from the electronic medical record and map it or otherwise electronically transfer it into a data field or fields in the XBRL medical record. The conversion engine further converts data associated with the data value into metadata. For example, for a value of 120, there would be associated with that value in the data field a plurality of metadata identifying the number 120 as a systolic blood pressure measurement, on a given pressure scale, and that the measurement was taken at 4 pm on Apr. 2, 2006. In one embodiment, the conversion engine could also form selected links in a link base between and/or among the data fields and between and among metadata for the different data fields, and between and among each component (elements, tuples, metadata, and other resources) based on a taxonomy. Alternatively, these links could be formed at a later time by means of programming a link.

Block 104 comprises an operation of extracting the data made over a period of time for that patient in a given data field and its associated metadata representing attributes or other related metadata for the data value. In one embodiment, this extracting operation comprises determining a period of time or sequence of events for the data from a data field to be accessed, e.g., blood pressure readings made over the last 2 years, or blood pressure readings made during the last 4 doctors visits. For an extraction based of blood pressure data over the last 2 years, the search engine for the repository would extract blood pressure data fields having metadata for date that meets a requirement of being measured in 2005 and 2006. Extraction can be initiated ad hoc by an individual physician or automatically by a software program and displayed and/or it can be analyzed.

Block 106 comprises an operation of performing an algorithm on the extracted data values and related attributes, metadata and other data values made over the period (of time or the sequence of events) for the at least one of the data fields to obtain an algorithm calculation result. In one embodiment, the data field may be the blood pressure data field, and the data values extracted may by the Systolic blood pressure measurements. The algorithm in this embodiment may be a regression algorithm. By way of example, the regression algorithm may be a single variant regression algorithm or a multi-variant regression algorithm. For the blood pressure embodiment, the regression algorithm would operate to take the plurality of blood pressure data values and associated metadata observed over time and calculate the rate of change over time and, in the case of a multi-variant regression algorithm, the rate of change as explained by other variables, e.g., r squared or other measures of disbursement around a calculated mean rate of change to obtain an algorithm calculation result. For example, what proportion of a rate of change (r squared) of blood pressure over a period of years is explained by sex alone (male/female)? What proportion of the blood pressure change is explained by body mass index (proportion over or under weight for a given height) or changes to the body mass? For example, using a body mass index, it can be determined that patient A is 0.95 of ideal and patient B is 1.20 of ideal for their respective heights and that this difference should correspond to a blood pressure change over time of a given amount. Additional variables might be smoker or non-smoker. Each of these variables can be calculated separately and their r squared for the proportion of the rate of change they represent estimated. In the multi-variant analysis, it is what r squared or proportion of an increase in calculated rate of increase are explained by two or more variants, body mass and smoking, for example. This combined two variable should have a higher r squared than either one singly. In effect a combination of variables can be used to explain the highest proportion (r squared) of the increase in value.

Block 108 comprises an operation of communicating the algorithm calculation results. The communication operation may be implemented by a presentation on a video display on any convenient electronic device, or via a communication of a text message, or a paper printout, or a printing or communication of a barcode, or via an email, or via a network communication, or a Web service, or an RFID tag, or any other mode of communication. The mode of communication is not limiting on the invention. In one embodiment, the communication may be made to video screen in a doctor's office. In another embodiment, the communication may be a network communication to an electronic database at a medical facility or payor facility or other appropriate facility.

It should be noted that the XBRL or equivalent medical record may also retain and/or include the original incoming records in electronic form. Accordingly, the XBRL or equivalent medical record may also include MRI data or X-ray data in electronic form, for example as an attachment. Alternatively, the XBRL or equivalent medical record may include one or more electronic links to the original records in one or more other electronic databases or repositories. Thus, the communication operation may include these medical records as an attachment, or may include links to the one or more original medical records.

In a further embodiment, an operation is provided of automatically identifying one or more potential diagnosis based on the algorithm calculation result, and communicating the potential diagnosis. By way of example, the potential diagnosis could be obtained by accessing one or more diagnosis databases and inputting the algorithm calculation result and retrieving a result. In one embodiment, the diagnosis database might be the John's Hopkins University data base on PSA.

In a further embodiment, an operation is provided of identifying one or more potential courses of treatment based on the algorithm calculation result, and communicating one or more of the potential courses of treatment. By way of example, the one or more potential courses of treatment could be obtained by accessing one or more medical treatment databases and inputting the algorithm calculation result and retrieving a result. For example, for metadata for blood pressure over 250 sys, one treatment that may be retrieved from the treatment database is prescribing the drug Lipitor (trademark of Pfizer Inc.) at a prescribed dosage. In one embodiment, the treatment database might comprise a pharmaceutical company's recommendation dosage from its database or website, or a treatment guidelines database from a third-party expert group; e.g. NIH or the American Heart Association, etc.

In a further embodiment, an operation is provided of determining a patient search profile for the patient based on one or more of body mass index, patient habits, race, and genotype, accessing one or more medical or genomic databases comprising treatment and/or medical outcomes, identifying one or more treatments and/or medical outcomes by the patient search profile, and communicating the one or more treatments and/or medical outcomes. The patient search profile would be obtained by selecting certain elements from the XBRL or equivalent medical record of the patient and inputting that search profile and the algorithm calculation results into a medical treatment and/or medical outcome databases, and communicating the results of the search using any convenient communication mode, such as a computer video display at a doctors office. In one embodiment, the system can match a type of drug and an amount of the drug to the human genome, e.g., for a given human genome, an amount of a drug, a preferred method of putting into the patient's system, its efficacy, and its potential side effect can be determined. Thus, a drug and a dosage that is optimum for the individual can be determined when coupled with his/her genomes that relate to how they process drugs. (See genotype below)

In a further embodiment, a series of patient measurements, such as, for example, blood pressure readings recorded in terms of metadata in the blood pressure data field of the XBRL or equivalent medical record made over two years, may be applied to regression algorithm as one variable, with age being another variable, and body mass or weight being a third variable. The results of the application of this regression algorithm on these three variables would provide a curve of patient A's blood pressure to body mass from, for example, ages 40-50. This curve could then be extrapolated to provide a blood pressure trend for a given body mass as patient A ages. Note that body mass could be considered to be a proxy for patient A's eating and exercise habits. Alternatively, a formula could be used to project patient A's blood pressure at age 60 or beyond. For example, a formula can be determined from blood pressure data collected over time from other people at various weights and ages to determine a rate of increase of blood pressure with age for a given body mass. Thus, using the formula, a current value of patient A's blood pressure, plus age and body mass can be used to calculate the rate of increase in blood pressure given the body mass for patient A in ten years. This projected blood pressure result can then be displayed alone, or in one embodiment, against observed outcomes for a person with this projected blood pressure and body mass obtained, for example, from medical databases or articles. This embodiment can be a powerful tool to help physicians persuade patients to (a) change life style (b) take medicine, etc. Thus, the use of the XBRL or equivalent medical record in combination with the one or more algorithms provides the ability to develop data from a selected data field over time, project data in the future, and display alone and/or along with medical outcomes for that profile group.

Medical outcomes might be determined as follows. In one example implementation, a patient search profile for the patient based on one or more of body mass index, patient habits, race, genotype is selected. Then one or more medical databases comprising data for a selected data field, e.g., blood pressure, for other people along with one or more of the other person's body mass index, patient habits, race, genotype are accessed, and the patient A current or a projected value for that selected data field along with the patient a A search profile (body mass, age) are input into the data base to obtain one or more medical outcomes. Such a medical outcome might comprise a statement that 40% of men with a body mass of X and a projected blood pressure of Y at age 50 will be deceased within the following 10 years. This medical outcome would then be communicated, as discussed previously. As another example, a doctor examining a patient having diabetes, heart disease, and having a smoking habit all together will have a different projected medical outcome compared to having only one trait. Using an appropriate outcome probability algorithm, a doctor may provide the advice that “We're 90% confident that a man with your three traits will die within ten years.” Such a communication would be made to motivate a change in the patient's habits.

Alternatively, instead of determining and communicating an average medical outcome of a profile group based on the patient's search profile, an average medical outcome of all patients that correlate with the algorithm calculation results may be communicated.

In yet a further embodiment, the XBRL medical record for the patient includes medical treatment records for a given episode, for example, such treatment records for the given episode might include a visit to a hospital for one or more days, the performance of a variety of tests to obtain a diagnosis, physician services for a treating physician and a radiologist, and the application of one or more treatments. In this embodiment, an operation of matching the medical treatment records of the patient for the given episode to insurance coverage, which may in one embodiment include a matching to covered categories and/or coverage limits and/or deductibles is performed to obtain match results. These match results are then electronically communicated. Thus, in one embodiment the medical treatment records for the given episode may comprise at least one selected from the group of cost of room, disease treatment, tests, and doctor fee. The matching step may be performed at patient checkout from a treatment location. Alternatively, the matching step may be performed by or for an insurance carrier providing the insurance coverage to the patient. The matching step may be automatic or triggered by an event, e.g. checkout or on request, e.g. by the attending physician.

In a yet further embodiment, a potential diagnosis is identified based on the algorithm calculation results. An insurance carrier associated with the patient and insurance coverage of that insurance carrier for one or more treatments for the potential diagnosis are determined. This insurance coverage for the one or more potential treatments under this insurance carrier may then be communicated. This communication mode is not limiting on the invention. However, in one embodiment, the communication may be displayed on a video screen in a physician's office or at a hospital.

In a yet further embodiment, at a trigger time and/or date automatically accessing the XBRL medical record for the patient, for at least one treatment and/or drug indicated in the accessed XBRL medical record as having been prescribed, extracting drug or treatment program data from one or more medical databases; and communicating the status of the drug or treatment data. In one embodiment, the extracting step may obtain the treatment and/or drug data posted to the medical database after a predetermined date. For example, if it is determined from the XBRL or equivalent medical record that Lipitor had been prescribed in August 2004, then the system may access one or more pharmaceutical databases from the Internet or other convenient network accessible electronic database and extract medical articles about Lipitor and its efficacy and/or side affects posted after August 2004, or after the most recent data extraction step has been performed for that drug for that patient. Note that this data extraction step may be performed every time the patient visits a doctor's office, or every time the XBRL or equivalent record is changed or a treatment is added to the record or changed.

In a further embodiment, a method is provided for determining fulfilling medical drug refills, comprising accessing a patient medical repository of patient medical records comprising data fields containing clinical examination data and/or laboratory results made over a period of time and associated metadata representing attributes at the data value level based on a medical taxonomy; selecting a given patient and determining that the patient has been prescribed or otherwise given a supply of medicine with a prescribed dosage; calculating or otherwise determining a first date when the supply of medicine will reach a predetermined level; and automatically on a second date generating a communication to at least one of the patient, a prescribing doctor, and a medicine supplier relating to the supply of medicine. For example, the system may determine that a patient has been given 30 day supply of medicine, and may automatically generate an e-mail (notice) to the patient and/or the prescribing doctor inquiring whether the patient has completed the treatment or not and requesting a response or reply e-mail. This can be repeated until the patient and/or doctor responds. Similarly, if new medicines are to be purchased or renewed, these can be flagged and reminders sent to the patient, physician, etc.

In a further embodiment, data indicating whether the patient took the prescribed drugs and for how long, e.g., stopped after 2 weeks, can be included within a data field or in its own separate data field. This data may be obtained via patient interviews or via laboratory measurements of the drug in the blood stream, or in any other convenient manner.

In a further embodiment, the system may automatically monitor one or more medical databases and extract new data relating to at least one treatment and/or drug indicated in the XBRL medical record of the patient as having been prescribed. Any new data would then be communicated relating to the at least one treatment and/or drug data.

In a yet further embodiment, in order to verify the conformance of the newly added data in the XBRL medical record to the original source, the steps may be performed of electronically communicating the metadata for the clinical examination data or laboratory results newly added or to be added to the medical repository to a medical source for the clinical examination data or laboratory results or its designee. The system would then look for receipt of validation data from the medical source or its designee that the metadata reflecting the medical test data and/or medical examination results is accurate.

In one embodiment, the steps of this invention are practiced on clinical examination data or laboratory results received from a plurality of different computer systems.

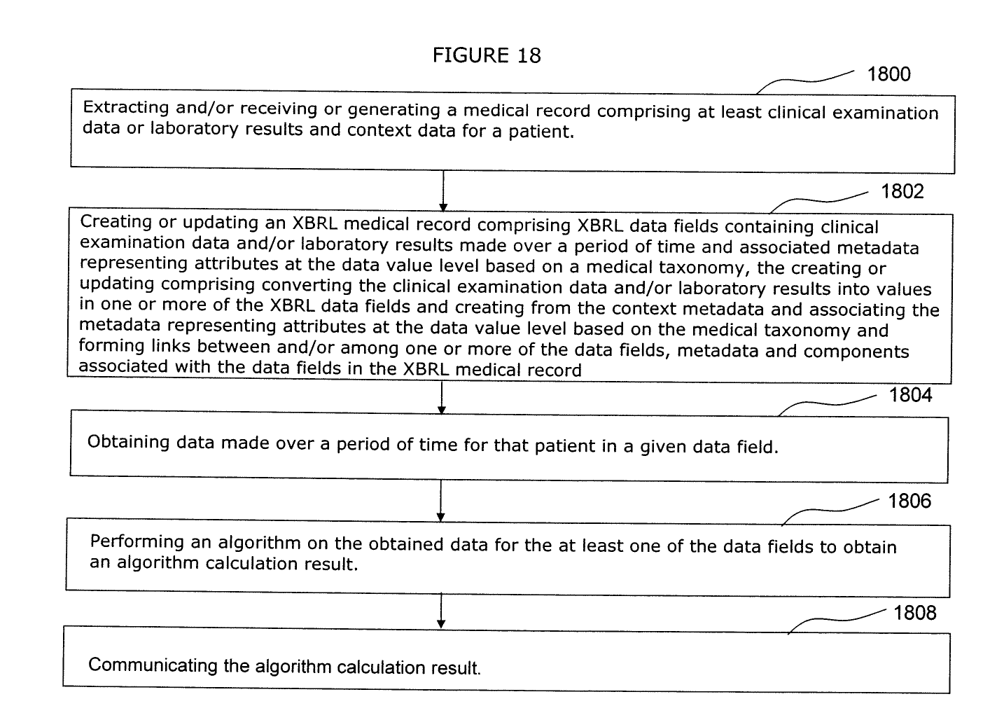

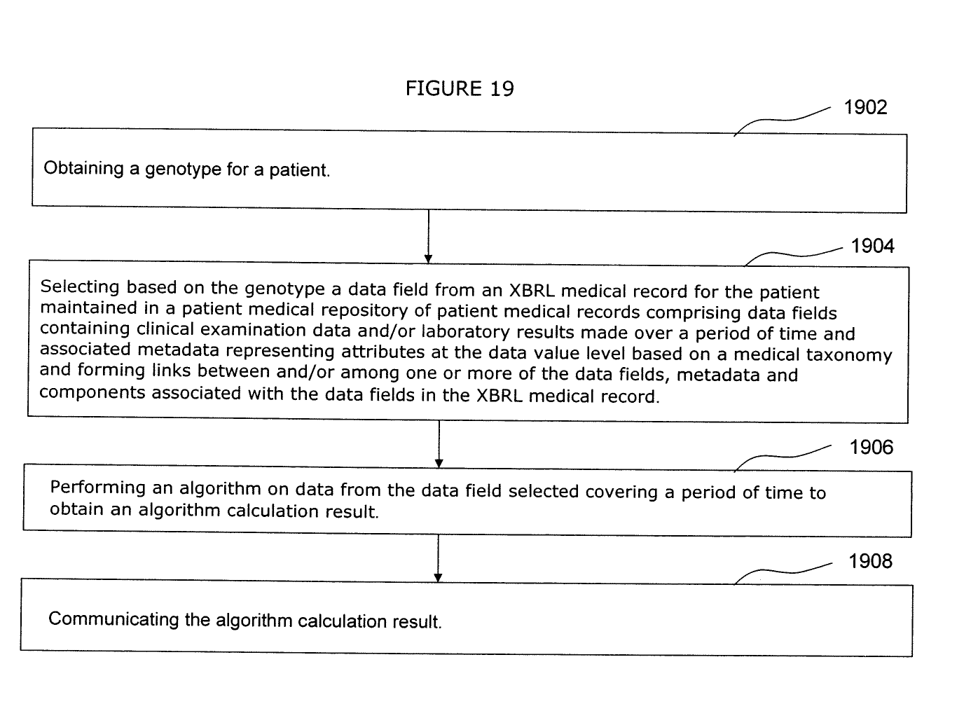

Referring to FIG. 18, a healthcare management method based on genotype is provided. In block 202, an operation is provided of obtaining a genotype and/or protean genotype for a patient. This operation may be performed by accessing an electronic database that includes the patient's genotype. Alternatively, the patient's genotype may be available in an XBRL medical record for the patient.

In block 204 an operation is performed of selecting based on the genotype a data field from an XBRL medical record for the patient, wherein the XBRL medical record contains clinical examination data and/or laboratory results in one or more data fields and metadata according to a medical taxonomy representing the values for the clinical examination data or laboratory results made over a period of time and a time the results were made. In one embodiment, this selection of the data field is accomplished by accessing one or more electronic databases and inputting the patient's genotype to identify from the data base one or more common diseases or conditions for that genotype. This identifying may include determining one or more symptoms or other markers for the disease or condition. For example, the data base may provide data indicating a predisposition (higher or lower) for a certain disease because of the presence or absence of a genotype and, if present, is it normal or mutated for a certain genotype is heart disease, high blood pressure, and list common markers for that disease, such as high blood pressure. Accordingly, a blood pressure data field may be selected.

In block 206 an algorithm is performed on data from the data field covering a period of time to obtain algorithm calculation results. For example, a regression algorithm that is single variant or multi-variant may be performed on the blood pressure data to project a given blood pressure value in the future.

In block 208, an operation is performed of communicating these algorithm calculation results, as described earlier.

In another embodiment, the algorithm performed on the data might comprise a comparison algorithm or a scoring based on a scoring protocol and summing algorithm.

In one embodiment, a further operation is performed of identifying from the algorithm calculation results whether markers are present that the patient is suffering or may in the future suffer from a particular disease. In one embodiment, this identifying operation may be performed by accessing one or more diagnosis databases and inputting the algorithm calculation result and retrieving a result. The data base selected may be determined by the particular data field being reviewed. In one embodiment, the diagnosis database might be the might be derived from secondary XBRL or non-XBRL data from patients with a similar condition. In another embodiment, the database may be from collected clinical data from clinical interviews or laboratory results. The results of this disease identification is then communicated.

In a further embodiment, secondary patient data comprised of actual patient medical data stripped of its identifying characteristics, can be aggregated and used to build a data base for treatments, projected outcomes.

In a further embodiment, the operations may be performed of identifying one or more potential courses of treatment based on the algorithm calculation result or the identified disease result; and communicating the one or more of the potential courses of treatment. By way of example, the one or more potential courses of treatment could be obtained by accessing one or more medical treatment databases and inputting the algorithm calculation result and or identified disease result and retrieving one or more potential courses of treatment. For example, for metadata for blood pressure over 250 sys, one treatment that may be retrieved from the treatment database may be prescribing the drug Lipitor (trademark of Pfizer Inc.) at a prescribed dosage.

In a further embodiment, an operation may be performed of identifying an insurance carrier associated with the patient and insurance coverage offered by the identified insurance carrier for one or more of the treatments for the disease result. This insurance coverage for the one or more potential courses of treatments would then be communicated.

In a further embodiment, an operation may be performed of determining a medicine dosage based at least in part on the genotype, and communicating the medicine dosage. For example, the communicating the medicine dosage operation may comprise displaying the medicine dosage on a video screen to medical personnel.

In a further embodiment, a method is provided for accurately prescribing medicine, comprising receiving/inputting prescription input data of a new medicine prescription or a refill; accessing a patient medical repository of patient medical records comprising data fields containing medicines prescribed over a period of years for the patient and associated diagnoses and associated metadata representing attributes at the data value level based on a medical taxonomy; processing or having processed the prescription input data, wherein the processing comprises one from the group of updating the patient medicine repository with the prescription input data, comparing the prescription input data to a measure of comparison, and a side effects database for prescription drugs, and communicating the prescription input data. By way of example, prescription input data comprising “2 mg Lipitor once a day for six months” would be sent from a doctors office to a patient medical repository containing some medical records for the patient in XBRL format. The “2 mg Lipitor” dosage could then be added to the XBRL patient medical record for that patient, or the dosage compared against a dosage standard for Lipitor obtained from a dosage database or a pharmaceutical database, or a database of side effects could be accessed and the most recent information on side effects from Lipitor downloaded. If the prescribed dosage was in an abnormal range as determined from a standard dosage obtained from the dosage database, e.g., well above or well below a recommended dosage, an alert could be generated. Note that a parameter of the dosage could also be genotype, to thereby determine a correct dosage for a given genotype. If the prescribed dosage is in a normal range, then the prescription input data could be communicated to a pharmacy, a doctor, the patient, a medical record, or other appropriate location or person. The communication could be by e-mail or using any other convenient mode of communication. The communication could also include the patient's demographic information, insurance information, and the physician's diagnosis. Thus, the system in one embodiment assists in (1) getting the prescription recorded in the XBRL medical record correctly and (2) getting it transmitted to the pharmacists correctly and in a standardized, application independent format. The system may include follow up checking on refills or reminders to be sure the patient is taking it, as set forth previously. This system would be very useful, particularly in situations where the doctors with Bluetooth or equivalent transmitters are making the prescription verbally, recording it electronically and transmitting it electronically. Note that the system is particularly advantageous because all of the prescriptions for the patient over the years are all together in one electronic XBRL record which could be sorted by date, medicine, disease, and e.g. high blood pressure.

Implementation of some embodiments of the invention using XBRL for interoperability may result in one or more of the following benefits to the health care professional and their patients.

Available Records
After a geographic move or referral from a primary care physician, a patient's full Multi-year medical records would be available instantly in electronic form. This minimizes the incomplete file syndrome where medical histories are taken again, lab work repeated, and other duplicative testing initiated, saving time, money, and increasing the probability of superior medical care.

Reduced Medical Errors
Reduction in medical errors from elimination of paper records and the ability to integrate a patient's clinical, laboratory, and pharmaceutical information into one integrated record would be immediately accessible will reduce medical errors from incomplete, lost, and non-related medical records Prescribing electronically will measurably reduce the well documented pharmacy errors due to illegible handwriting and permit automatic analysis of potential interactions with previously prescribed medicines.

Reduced Costs
Accessible patient records would minimize completing duplicate forms, redundant examinations and laboratory tests. Transfer of patient records from a primary care physician to a specialist, to another specialists or hospital would be as seamless as using e-mail. This reduces today's duplicate and often redundant forms, tests, and x-rays. It makes moving from one doctor to another or one city to another easier and ensures that a continuity of records is available to the new physician.

Strengthened HIPAA Enforcement
In some embodiments, the present XBRL invention need not alter or affect a physician's HIPAA a priori medical records policy or security practices. However, it can be used to facilitate programs to generate an “electronic audit trail by data field” of who accessed, removed, or altered a with a patient's medical record. For example, if an intruder took or altered a patient's HIV score, there can be a record for that data field. XBRL has the capability to create an electronic record of who accessed the electronic medical record, when they did it, and what data fields they altered. This electronic capability can materially strengthen HIPAA enforcement efforts, increase civil judgments and penalties. This includes consideration of appropriately more severe criminal penalties, including incarceration for individuals who knowingly breach the sanctity a patient's electronic medical record for profit. Paper records and XML do not and can never provide a comparable electronic audit trail feature for HIPAA enforcement.

New Feasible Diagnostic Tools
Using XBRL, data within multi-year clinical and laboratory records can be accessed and analyzed. A patient's XML medical record is essentially a picture of the printed page designed to be viewed by humans. It can be viewed, but the data in it is inactive. It must be extracted, transferred, and entered into a new computer program. With XBRL software can monitor changes in clinical or laboratory values from year to year within the medical record's data field without removing it from the patient's file. It is “Interactive.”

It should be noted that although the flow charts provided herein show a specific order of method steps, it is understood that the order of these steps may differ from what is depicted. Also two or more steps may be performed concurrently or with partial concurrence. Such variation will depend on the software and hardware systems chosen and on designer choice. It is understood that all such variations are within the scope of the invention. Likewise, software and web implementations of the present invention could be accomplished with standard programming techniques with rule based logic and other logic to accomplish the various database searching steps, correlation steps, comparison steps and decision steps. It should also be noted that the word “component” as used herein and in the claims is intended to encompass implementations using one or more lines of software code, and/or hardware implementations, and/or equipment for receiving manual inputs.

The foregoing description of embodiments of the invention has been presented for purposes of illustration and description. It is not intended to be exhaustive or to limit the invention to the precise form disclosed, and modifications and variations are possible in light of the above teachings or may be acquired from practice of the invention. The embodiments were chosen and described in order to explain the principals of the invention and its practical application to enable one skilled in the art to utilize the invention in various embodiments and with various modifications as are suited to the particular use contemplated.

What is claimed is:

1. A healthcare management method, comprising:
extracting and/or receiving or generating a medical record comprising at least clinical examination data or laboratory results and context data therefore for a patient;
creating or updating an XBRL medical record comprising XBRL data fields containing clinical examination data and/or laboratory results made over a period of time and associated metadata representing attributes at the data value level based on a medical taxonomy, the creating or updating comprising converting the clinical examination data and/or laboratory results into values in one or more of the XBRL data fields and creating from the context metadata and associating the metadata representing attributes at the data value level based on the medical taxonomy and forming links between and/or among at least two items selected from one or more of the categories of categories of the data fields, metadata and components associated with the data fields in the XBRL medical record;
obtaining data made over a period of time for that patient in a given data field;
performing an algorithm on the obtained data for the at least one of the data fields to obtain an algorithm calculation result; and
communicating the algorithm calculation result.

2. The method as defined in claim 1, wherein the algorithm is a regression algorithm.

3. The method as defined in claim 2, wherein the regression algorithm is a single variant regression algorithm.

4. The method as defined in claim 2, wherein the regression algorithm is a multi-variant regression algorithm.

5. The method as defined in claim 1, further comprising:
automatically identifying one or more potential diagnosis based on the algorithm calculation results; and
communicating the potential diagnosis.

6. The method as defined in claim 1, further comprising:
identifying one or more potential courses of treatment based on the algorithm calculation results; and
communicating the potential course of treatment.

7. The method as defined in claim 1, further comprising:
determining a patient search profile for the patient based on one or more of body mass index, patient habits, race, genotype;
automatically accessing one or more medical databases comprising treatment and/or medical outcomes and identifying and selecting one or more treatments and/or medical outcomes for the patient search profile; and
communicating the one or more treatments and/or medical outcomes.

8. The method as defined in claim 7, wherein the communicating the one or more treatments and/or medical outcomes comprises displaying the one or more treatments and medical outcomes correlated with those treatments.

9. The method as defined in claim 1, wherein the clinical examination results or laboratory results are attached to or linked to the XBRL medical record.

10. The method as defined in claim 1, further comprising:
determining a patient search profile for the patient based on one or more of body mass index, patient habits, race, genotype;
automatically accessing one or more medical databases comprising one or more medical outcomes based on the algorithm calculation results and the patient search profile; and
communicating the one or more medical outcomes.

11. The method as defined in claim 1,
accessing one or more medical databases comprising treatment and/or medical outcomes and selecting one or more treatments and/or medical outcomes based on the algorithm calculation results; and
communicating the one or more of the treatments and/or medical outcomes.

12. The method as defined in claim 11, wherein the communicating the one or more of the treatments and/or medical outcomes step comprises displaying the one or more of the treatments and/or medical outcomes.

13. The method as defined in claim 11, wherein an average medical outcome of a profile group is communicated for a given algorithm calculation result or projected result.

14. The method as defined in claim 11, wherein an average medical outcome of all patients that correlate with the algorithm calculation results is communicated for a given algorithm calculation result or projected result.

15. The method as defined in claim 1, further comprising:
automatically determining a potential diagnosis based on the algorithm calculation results;
determining an insurance carrier associated with the patient and insurance coverage for one or more treatments for the potential diagnosis; and
communicating the potential diagnosis and the insurance coverage for the one or more potential treatments.

16. The method as defined in claim 1, further comprising:
at a trigger time and/or date automatically accessing the XBRL medical record for the patient;
for at least one treatment and/or drug indicated in the accessed XBRL medical record as having been prescribed, extracting drug or treatment data from one or more medical databases; and
communicating the drug or treatment data.

17. The method as defined in claim 16, wherein the extracting step obtains the treatment and/or drug data posted to the medical database after a predetermined date.

18. The method as defined in claim 17, wherein the predetermined date is a date of a previous visit data extraction step performed for that drug for that patient or a date of a last change to the XBRL record.

19. The method as defined in claim 1, further comprising:
automatically monitoring one or more medical databases new data relating to at least one treatment and/or drug indicated in the XBRL medical record of the patient as having been prescribed; and
communicating the new data relating to the at least one treatment and/or drug data.

20. The method as defined in claim 1, further comprising:
electronically communicating the data for medical test data and/or medical examination results newly added or to be added to the XBRL medical record to a medical source for the medical test data and/or medical examination results or its designee; and
receiving validation data from the medical source or its designee that the data reflecting the medical test data and/or medical examination results is accurate.

21. The method as defined in claim 1, further comprising performing the steps of claim 1 on new clinical examination data or laboratory results received from a plurality of different computer systems.

22. The method as defined in claim 1, wherein the XBRL medical record for the patient includes medical treatment records for a given episode, and further comprising:
matching the medical treatment records of the patient for the given episode to insurance coverage to obtain match results; and
electronically communicating the match results.

23. The method as defined in claim 22, wherein medical treatment records for the given episode comprise at least one selected from the group of cost of room, disease treatment, tests, and doctor fee, and further comprising performing the matching step at patient checkout from a treatment location.

24. The method as defined in claim 22, further comprising performing the matching step on a periodic basis, and wherein the communicating the match results step comprises communicating the results to an insurance carrier providing the insurance coverage.

25. A method for creating a or repository of medical treatments and/or medical outcomes, comprising:
stripping or having stripped patient-identifying characteristics from patient medical data in the XBRL medical records to obtain stripped XBRL medical data;
aggregating the stripped XBRL medical data with other stripped XBRL medical data into medical treatment and/or medical outcomes repository; and
granting electronic access to the medical treatment and/or medical outcomes repository.

26. A method for determining fulfilling medical drug refills, comprising
accessing XBRL patient medical records comprising data fields containing clinical examination data and/or laboratory results made over a period of time and associated metadata representing attributes at the data value level based on a medical taxonomy;
selecting a given patient and determining that the patient has been prescribed or otherwise given a supply of medicine with a prescribed dosage;
calculating or otherwise determining a first date when the supply of medicine will reach a predetermined level; and
automatically on a second date generating a communication to at least one of the patient, a prescribing doctor, and a medicine supplier relating to the supply of medicine.

27. The method as defined in claim 26, wherein the second date is a date a prescribed number of days before the first date.

28. The method as defined in claim 26, wherein the communication is by an e-mail or text message inquiring whether the patient has completed the treatment and requesting a response or reply e-mail.

29. The method as defined in claim 26, wherein the communication is a reminder to renew a prescription or obtain a refill of the medicine.

30. A healthcare management method based on genotype, comprising:
obtaining a genotype for a patient;
selecting based on the genotype a data field from an XBRL medical record for the patient maintained in a patient medical repository of patient medical records comprising data fields containing clinical examination data and/or laboratory results made over a period of time and associated metadata representing attributes at the data value level based on a medical taxonomy and forming links between and/or among at least two items selected from one or more of the categories of the data fields, metadata and components associated with the data fields in the XBRL medical record;
performing an algorithm on data made over a period of time for at least one of the data fields to obtain an algorithm calculation result; and
communicating the algorithm calculation result.

31. The method as defined in claim 30, wherein the algorithm is a regression algorithm.

32. The method as defined in claim 30, wherein the algorithm is a comparison algorithm.

33. The method as defined in claim 30, further comprising:
determining from the algorithm calculation results whether indicators are present that the patient is suffering or may in the future suffer from a particular disease; and
communicating a disease result from this determining step.

34. The method as defined in claim 33, further comprising:
determining one or more potential courses of treatment based on the disease result; and
communicating the one or more potential courses of treatment.

35. The method as defined in claim 34, further comprising:
determining an insurance carrier associated with the patient and insurance coverage for one or more treatments for the disease result; and
communicating the insurance coverage for the one or more potential courses of treatments.

36. The method as defined in claim 30, further comprising:
determining a medicine dosage based at least in part on the genotype; and
communicating the medicine dosage.

37. The method as defined in claim 36, wherein the communicating the medicine dosage comprises displaying the medicine dosage.

38. A method for prescribing medicine, comprising
receiving or inputting prescription input data of a new medicine prescription or a refill;
accessing XBRL patient medical record comprising data fields containing medicines prescribed over a period of years for the patient and associated diagnoses and associated metadata representing attributes at the data value level based on a medical taxonomy;
processing or having processed the prescription input data, wherein the processing comprises one from the group of updating the XBRL patient medicine record with the prescription input data, comparing the prescription input data to a measure of comparison, and a side effects database for prescription drugs, and
communicating the prescription input data.

39. The method as defined in claim 38, wherein the medical taxonomy includes one or more predetermined dosages or a range of dosages for a given medicine referenced in the prescription input data, and wherein the dosage in the prescription input data for the given medicine is not entered if it is not one of the one or more predetermined dosages or within the prescribed range without an exception.

40. A healthcare management system, comprising:
a data repository; and
one or more processors operably connected to the data repository for implementing the following components:
a component for extracting and/or receiving or generating a medical record comprising at least clinical examination data or laboratory results and context data therefore for a patient;
a component for creating or updating an XBRL medical record comprising XBRL data fields containing clinical examination data and/or laboratory results made over a period of time and associated metadata representing attributes at the data value level based on a medical taxonomy, the creating or updating comprising converting the clinical examination data and/or laboratory results into values in one or more of the XBRL data fields and creating from the context metadata and associating the metadata representing attributes at the data value level based on the medical taxonomy and forming links between and/or among at least two items selected from one or more of the categories of categories of the data fields, metadata and components associated with the data fields in the XBRL medical record;
a component for obtaining data made over a period of time for that patient in a given data field;
a component for performing an algorithm on the obtained data for the at least one of the data fields to obtain an algorithm calculation result; and
a component for communicating the algorithm calculation result.

41. A healthcare management program product, comprising:
one or more computer usable media having computer readable program code embodied therein or among them, to be executed by a computer, the computer readable program code comprising:
program code for extracting and/or receiving or generating a medical record comprising at least clinical examination data or laboratory results and context data therefore for a patient;
program code for creating or updating an XBRL medical record comprising XBRL data fields containing clinical examination data and/or laboratory results made over a period of time and associated metadata representing attributes at the data value level based on a medical taxonomy, the creating or updating comprising converting the clinical examination data and/or laboratory results into values in one or more of the XBRL data fields and creating from the context metadata and associating the metadata representing attributes at the data value level based on the medical taxonomy and forming links between and/or among at least two items selected from one or more of the categories of categories of the data fields, metadata and components associated with the data fields in the XBRL medical record;
program code for obtaining data made over a period of time for that patient in a given data field;
program code for performing an algorithm on the obtained data for the at least one of the data fields to obtain an algorithm calculation result; and
program code for communicating the algorithm calculation result.

42. A healthcare management system based on genotype, comprising:
a data repository; and
one or more processors operably connected to the data repository for implementing the following components:
a component for obtaining a genotype for a patient;
a component for selecting based on the genotype a data field from an XBRL medical record for the patient maintained in a patient medical repository of patient medical records comprising data fields containing clinical examination data and/or laboratory results made over a period of time and associated metadata representing attributes at the data value level based on a medical taxonomy and forming links between and/or among at least two items selected from one or more of the categories of the data fields, metadata and components associated with the data fields in the XBRL medical record;
a component for performing an algorithm on data made over a period of time for at least one of the data fields to obtain an algorithm calculation result; and
a component for communicating the algorithm calculation result.

43. A healthcare management program product based on genotype, comprising:
one or more computer usable media having computer readable program code embodied therein or among them, to be executed by a computer, the computer readable program code comprising:
program code for obtaining a genotype for a patient;
program code for selecting based on the genotype a data field from an XBRL medical record for the patient maintained in a patient medical repository of patient medical records comprising data fields containing clinical examination data and/or laboratory results made over a period of time and associated metadata representing attributes at the data value level based on a medical taxonomy and forming links between and/or among at least two items selected from one or more of the categories of the data fields, metadata and components associated with the data fields in the XBRL medical record;
program code for performing an algorithm on data made over a period of time for at least one of the data fields to obtain an algorithm calculation result; and
program code for communicating the algorithm calculation result.

44. A system for creating a or repository of medical treatments and/or medical outcomes, comprising:
a data repository; and
one or more processors operably connected to the data repository for implementing the following components:
a component for stripping or having stripped patient-identifying characteristics from patient medical data in the XBRL medical records to obtain stripped XBRL medical data;
a component for aggregating the stripped XBRL medical data with other stripped XBRL medical data into medical treatment and/or medical outcomes repository; and
a component granting electronic access to the medical treatment and/or medical outcomes repository.

45. A program product for creating a or repository of medical treatments and/or medical outcomes, comprising:
one or more computer usable media having computer readable program code embodied therein or among them, to be executed by a computer, the computer readable program code comprising:
program code for stripping or having stripped patient-identifying characteristics from patient medical data in the XBRL medical records to obtain stripped XBRL medical data;
program code for aggregating the stripped XBRL medical data with other stripped XBRL medical data into medical treatment and/or medical outcomes repository; and
program code for granting electronic access to the medical treatment and/or medical outcomes repository.

46. A system for determining fulfilling medical drug refills, comprising
a data repository; and
one or more processors operably connected to the data repository for implementing the following components:
a component for accessing XBRL patient medical records comprising data fields containing clinical examination data and/or laboratory results made over a period of time and associated metadata representing attributes at the data value level based on a medical taxonomy;
a component for selecting a given patient and determining that the patient has been prescribed or otherwise given a supply of medicine with a prescribed dosage;
a component for calculating or otherwise determining a first date when the supply of medicine will reach a predetermined level; and
a component for automatically on a second date generating a communication to at least one of the patient, a prescribing doctor, and a medicine supplier relating to the supply of medicine.

47. A program product for determining fulfilling medical drug refills, comprising:
one or more computer usable media having computer readable program code embodied therein or among them, to be executed by a computer, the computer readable program code comprising:
program code for accessing XBRL patient medical records comprising data fields containing clinical examination data and/or laboratory results made over a period of time and associated metadata representing attributes at the data value level based on a medical taxonomy;
program code for selecting a given patient and determining that the patient has been prescribed or otherwise given a supply of medicine with a prescribed dosage;
program code for calculating or otherwise determining a first date when the supply of medicine will reach a predetermined level; and
program code for automatically on a second date generating a communication to at least one of the patient, a prescribing doctor, and a medicine supplier relating to the supply of medicine.

48. A system for accurately prescribing medicine, comprising
a data repository; and
one or more processors operably connected to the data repository for implementing the following components:
a component for receiving or inputting prescription input data of a new medicine prescription or a refill;
a component for accessing XBRL patient medical record comprising data fields containing medicines prescribed over a period of years for the patient and associated diagnoses and associated metadata representing attributes at the data value level based on a medical taxonomy;
a component for processing or having processed the prescription input data, wherein the processing comprises one from the group of updating the XBRL patient medicine record with the prescription input data, comparing the prescription input data to a measure of comparison, and a side effects database for prescription drugs, and
a component for communicating the prescription input data.

49. A program product for prescribing medicine, comprising
one or more computer usable media having computer readable program code embodied therein or among them, to be executed by a computer, the computer readable program code comprising:
program code for receiving or inputting prescription input data of a new medicine prescription or a refill;
program code for accessing XBRL patient medical record comprising data fields containing medicines prescribed over a period of years for the patient and associated diagnoses and associated metadata representing attributes at the data value level based on a medical taxonomy;
program code for processing or having processed the prescription input data, wherein the processing comprises one from the group of updating the XBRL patient medicine record with the prescription input data, comparing the prescription input data to a measure of comparison, and a side effects database for prescription drugs, and
program code for communicating the prescription input data.
